(19) United States
(12) Patent Application Publication
(10) Pub. No.: US 2026/0306592 A1
(43) Pub. Date: Oct. 8, 2026

(54) HUMAN FACTOR VII DELETION CONSTRUCTS FOR TREATING CANCER

(71) Applicant: THE CLEVELAND CLINIC FOUNDATION, Cleveland, OH (US)

(72) Inventors: Jeongwu LEE, Cleveland, OH (US); Hye-Min JEON, Cleveland, OH (US)

(21) Appl. No.: 19/479,862

(22) PCT Filed: Apr. 29, 2024
(86) PCT No.: PCT/US2024/026844

§ 371 Date: Oct. 29, 2025

Related U.S. Application Data
(60) Provisional application No. 63/500,076, filed on May 4, 2023.

(51) Int. Cl. A61K 38/48; A61K 31/495; A61K 45/06; A61P 35/00

(52) U.S. Cl. CPC A61K 38/4846; A61K 31/495; A61K 45/06; A61P 35/00; C12Y 304/21021

(57) ABSTRACT

Provided herein are compositions, kits, and methods for treating a human subject with cancer (e.g., brain cancer) with a composition comprising: a human delta Factor VII construct, or nucleic acid sequence encoding said human delta Factor VII construct. In certain embodiments, the human delta Factor VII construct comprises a Propeptide domain and EGF-1 domain, or portions thereof, and lacks a functioning Peptidase S1 domain. In some embodiments, the human subject has glioblastoma and has been previously treated with radiation therapy.

### CROSS-REFERENCE TO RELATED APPLICATIONS

The present application claims priority to U.S. provisional application Ser. No. 63/500,076, filed May 4, 2023, which is herein incorporated by reference in its entirety.

### STATEMENT REGARDING FEDERAL FUNDING

This invention was made with government support under CA223370 awarded by the National Institutes of Health. The government has certain rights in the invention.

### SEQUENCE LISTING

The text of the computer readable sequence listing filed herewith, titled “CCF-41795_601_SequenceListing.xml,” created on Apr. 29, 2024, having a file size of 9,956 bytes, is hereby incorporated by reference in its entirety.

### FIELD OF THE INVENTION

Provided herein are compositions, kits, and methods for treating a human subject with cancer (e.g., brain cancer) with a composition comprising: a human delta Factor VII construct, or nucleic acid sequence encoding said human delta Factor VII construct. In certain embodiments, the human delta Factor VII construct comprises a Propeptide domain and EGF-1 domain, or portions thereof, and lacks a functioning Peptidase S1 domain. In some embodiments, the human subject has glioblastoma and has been previously treated with radiation therapy.

### BACKGROUND OF THE INVENTION

GBM is the most lethal brain cancer with no curative therapies available. Recent anti-angiogenic and immunotherapy approaches have not yet shown durable clinical benefits for GBM patients (Gilbert et al., 2014; Lim et al., 2018). Currently, maximal surgical resection followed by radiotherapy (RT) and temozolomide (TMZ) chemotherapy is standard-of-care therapy for newly diagnosed GBM patients (Stupp et al., 2005). Limited benefits from current therapies can be attributed to multiple factors, including inherent radio-resistance of GBM tumors, preferential survival of GBM stem-like cells, and treatment-induced activation of pro-tumorigenic adaptive pathways (Bao et al., 2006; Barker et al., 2015; Chen et al., 2012).

Radiation therapy (RT) exerts cytostatic and cytotoxic anti-tumor effects against GBM, but tumor relapse is almost inevitable. Following RT, GBM tumors undergo phenotypic transition via global remodeling of epigenetic and transcriptome landscapes. RT enhances the secretion of various chemokines, cytokines, and ECM molecules by tumor cells (Winchell et al., 1964; Yoo et al., 2018). Many of RT-induced chemo/cytokines are known to induce GBM cell state transition and mediate reciprocal crosstalk between GBM cells and immune cells (Bhat et al., 2013; Halliday et al., 2014; Hara et al., 2021). In addition, RT is a potent inducer of coagulopathy and immune modulation (Ahmed et al., 2013; Kennedy et al., 2016; Magnus et al., 2013). While the above RT-induced global changes have long been hypothesized to account for tumor evolution and aggressiveness of recurrent tumors, the upstream initiators of these processes are poorly understood.

In many malignant tumors such as GBM, irradiation and targeted inhibitors transiently induce tumor cell senescence, referred to as therapy-induced senescence (TIS) (Ewald et al., 2010; Schmitt et al., 2022). Because the senescence program involves global epigenomic reprogramming and the elevated secretion of multiple proteins, referred to as the senescence-associated secretory phenotype (SASP), TIS may play a role in RT-induced global changes (Gorgoulis et al., 2019; He and Sharpless, 2017). Tumor cell senescence remains poorly defined, with key questions unanswered including how senescent tumor cell states are regulated and how TIS affects treatment resistance and tumor recurrence.

### SUMMARY OF THE INVENTION

Provided herein are compositions, kits, and methods for treating a human subject with cancer (e.g., brain cancer) with a composition comprising: a human delta Factor VII construct, or nucleic acid sequence encoding said human delta Factor VII construct. In certain embodiments, the human delta Factor VII construct comprises a Propeptide domain and EGF-1 domain, or portions thereof, and lacks a functioning Peptidase S1 domain. In some embodiments, the human subject has glioblastoma and has been previously treated with radiation therapy.

In some embodiments, provided herein are compositions comprising: a human delta Factor VII construct, or nucleic acid sequence encoding the human delta Factor VII construct, wherein the human delta Factor VII construct comprises: a) a human Factor VII Propeptide domain, or portion thereof with substantially the same activity (e.g., such a portion can be tested in Example 1 in place of the human Factor VII Propeptide domain tested therein, where, for example, truncation mutants can be tested until the smallest size that still works is found), b) a human Factor VII EGF-1 domain, or portion thereof with substantially the same activity (e.g., such a portion can be tested in Example 1 in place of the human Factor VII EGF-1 domain tested therein, where, for example, truncation mutants can be tested until the smallest size that still works is found), and wherein the human delta Factor VII construct lacks a Peptidase S1 domain or lacks a functional Peptidase S1 domain, wherein optionally the human delta Factor VII construct is less than 225 amino acids in length (e.g., less than 200 . . . 190 . . . or 180).

In certain embodiments, provided herein are methods of treating cancer comprising: treating a human subject with the delta Factor VII construct, or cell or expression vector containing the nucleic acid sequence encoding the delta Factor VII construct, as recited above or anywhere herein, wherein the human subject has cancer.

In particular embodiments, the human subject has brain cancer. In other embodiments, the human subject has glioblastoma (GBM). In additional embodiments, the subject has been subject to radiation therapy for cancer prior to the treating. In other embodiments, the human subject has GBM cells, and wherein the treating reduces the levels of F3 in the GBM cells.

In some embodiments, the treating comprises intravenous administration (or oral administration or intracranial). In further embodiments, the intravenous administration is in the neck of the human subject. In other embodiments, the treating reduces the level of F3 proteins in cancer cells of the subject. In particular embodiments, the treating induces ubiquitin-mediated degradation of human F3 proteins in cancer cells of the subject.

In other embodiments, the human delta Factor VII construct: i) binds human coagulation factor III (F3), ii) lacks pro-coagulation activity in an in vitro coagulation assay, and/or iii) induces ubiquitin-mediated degradation of human F3 proteins in cell culture. In additional embodiment, the human Factor VII Propeptide domain comprises the amino acid sequence of SEQ ID NO:6, or at least 30 consecutive amino acids (e.g., 30 . . . 33 . . . 35 or more) from SEQ ID NO:6, or SEQ ID NO:6 with one, two, three, four, or five amino acid deletions or conservative amino acid changes. In additional embodiments, the human Factor VII EGF-1 domain comprises the amino acid sequence of SEQ ID NO:8, or at least 30 consecutive amino acids from SEQ ID NO:8, or SEQ ID NO:8 with one, two, three, four, or five amino acid deletions or conservative amino acid changes.

In additional embodiments, the human delta Factor VII construct further comprises: c) a human Factor VII GLA domain, or portion thereof with substantially the same activity (e.g., such a portion can be tested in Example 1 in place of the human Factor VII GLA domain tested therein, where, for example, truncations mutants can be tested until the smallest size that still works is found). In some embodiments, the human Factor VII GLA domain comprises the amino acid sequence of SEQ ID NO:7, or at least 30 consecutive amino acids (e.g., at least 30 . . . 33 . . . 35 or more) from SEQ ID NO:7, or SEQ ID NO:7 with one, two, three, four, or five amino acid deletions or conservative amino acid changes. In particular embodiments, the human delta Factor VII construct further comprises: c) a human Factor VII EGF-2 domain, or portion thereof with substantially the same activity. In certain embodiments, the human Factor VII EGF-2 domain comprises the amino acid sequence of SEQ ID NO:9, or at least 30 consecutive amino acids (e.g., at least 30 . . . 33 . . . 35 or more) from SEQ ID NO:9, or SEQ ID NO:9 with one, two, three, four, or five amino acid deletions or conservative amino acid changes. In additional embodiments, the human delta Factor VII construct further comprises: c) a human Factor VII GLA domain, or portion thereof with substantially the same activity, and d) a human Factor VII EGF-2 domain, or portion thereof with substantially the same activity (e.g., such a portion can be tested in Example 1 in place of the human Factor VII EGF-2 domain tested therein, where truncations mutants, for example, can be tested until the smallest size that still works is found).

In some embodiments, the composition comprises the nucleic acid sequence, which is optionally present in an expression vector. In certain embodiments, the composition further comprises a neuro progenitor cell, and wherein the nucleic acid sequence is present in the neuro progenitor cells. In other embodiments, the compositions further comprise (or the subject is further administered) at least one of the following: Temozolomide (TMZ), an EGFR inhibitor, a MET inhibitor, and an AKT inhibitor. In other embodiments, the compositions further comprise at least one of the following: a physiologically tolerable buffer, normal saline, lactated ringers, and dextrose solution.

In additional embodiments, the composition comprises the human delta Factor VII construct. In further embodiments, the human delta Factor VII construct comprises SEQ ID NO: 3, 4, or 5.

In some embodiments, provided herein is a kit or system comprising: a) a composition as described herein, and b) an intracranial, or intravenous, therapeutic delivery device, a syringe vial, or shipment container. In some embodiments, the composition is present in the intracranial or intravenous therapeutic delivery device, or the syringe vial, or the shipment container.

In some embodiments, the human subject has glioblastoma (GBM). In other embodiments, the subject has been subject to radiation therapy for cancer prior to the treating. In additional embodiments, the human subject has GBM cells, and wherein the treating reduces the levels of F3 in the GBM cells.

### DESCRIPTION OF THE FIGURES

FIG. 1. Radiation-induced SA-βGal+ GBM cells clonally expand in vivo. (A) Representative images of SA-βGal activity in the brain sections. GBM tumor-bearing mice were not irradiated or irradiated (naïve and RT, respectively) and stained with X-gal or C12FDG five days later. Tumor regions were shown in H&E images. Different patient-derived GBMs are designated as numbers. (B) Proportion of RFP+ cells (human GBM cells) and RFP− cells (mouse cells) within C12FDG+ populations (n=3 per each). (C) Immunoblots of DNA damage response (DDR) proteins in sorted RT-C12FDG+ and C12FDG− GBM subpopulations. β-actin was used as a loading control. (D) In vitro clonogenic growth of irradiated GBM subpopulations. Single cells from each subpopulation were plated in soft agar, cultured for 3 weeks, and resultant colonies counted. (E) Flow cytometry plot of C12FDG and Nestin staining in naïve and RT GBM cells. Quantitation of C12FDG+/Nestin+ cells are shown. (F) Immunofluorescence (IF) staining images of C12FDG and senescence markers (HP1γ and H3K9me3). (G) Flow cytometry plot of H3K9me3 and Nestin staining in naïve, RT-bulk, and RT-C12FDG+ GBM cells. (H) t-distributed Stochastic Neighbor Embedding (t-SNE) plots of GBM single cells in the naïve, RT-bulk, and RT-C12FDG+ GBM cells (total of 105,653 cells). Color gradient was overlaid with stemness signature scores. (I) Immunoblots of H3K9me3 and GBM stemness markers (Nestin and Sox2) proteins in three matched sets of RT-C12FDG+ and RT-C12FDG− GBM subpopulations. (J to L) Barcode-mediated clonal expansion and clonal diversity determination analysis. (J) Experimental schematic. (K) Floating bar plots of RFP-labeled cell populations in vivo. Center line in each bar represents mean value (n=4 animals per group). (L) Numbers of unique barcodes and barcode distribution within the tumors derived from the indicated cell populations were determined by barcode index sequencing. (M) In vivo limiting dilution tumor formation results using naïve, RT-bulk, and RT-C12FDG+ GBM cells. Results are presented as mean±SD. * P<0.001.

FIG. 2. F3, highly expressed in irradiated C12FDG+ GBM cells, is associated with sternness, cell state transition, and an enhanced secretory phenotype. (A) A schematic of the cell surface marker screen. (B) The lists of the cell surface proteins that were enriched in RT-bulk and RT-C12FDG+ GBM cells compared to naïve cells. p<0.001. (C) IF staining images of C12FDG and F3 in the brains of PDX tumor-bearing mice. C12FDG+/F3+ double-positive cells were quantitated (n=3 per each tumor). (D and E) In vivo proliferation of RT bulk, RT-F3+, and RT-F3− cell populations (D) and in vivo limiting dilution tumor formation results. Refer to FIG. 1 K and M. (F) Enriched transcription factor-binding motifs in RT-F3+ subpopulations, as determined by ATAC sequencing analysis. (G and H) Immunoblots of pp65, pIκBα, EZH2, active-β-catenin, Sox2, and HUTS4 proteins in the RT-F3+ and RT-F3− GBM subpopulations. For detection with HUTS4 antibody (specific for the activated form of ITGB1), non-denaturing gels were used. (I) Quantitation of stemness gene set expression in naïve, RT-bulk, RT-F3+, and RT-F3− GBM single cells (total of 28,890 cells). (J) Immunoblots of MES GBM transition-associated proteins (pSTAT3, IL6, and YKL-40). (K) IF images of F3 and CD44 in matched naïve and irradiated GBM tissue slices and quantitation. (L) Quantitation of SASP factor gene set expression in the above subpopulations. (M) Heatmap plots and quantitation of the secreted proteins from naïve, RT-bulk, and RT-F3+ GBM cells. Results are mean±SD. * p<0.001.

FIG. 3. Radiation-induced F3 prime global changes both in tumor and microenvironment. (A) Immunohistochemical (IHC) staining images of F3, fibrin, and YKL-40 in 827 GBM tumors. Tumor-bearing mice were irradiated in vivo and tumors were harvested 5 days later. (B and C) IF images of fibrin, FN1 (an ECM molecule), F4/80, CD163 (M2-like TAM) staining. Fibrin+/FN1+ areas and numbers of YKL-40+, F4/80+, and CD163+ cells were quantitated (n=4 per each). (D to F) IF images of fibrin, IBA1, CD163, CD44, F3 in syngeneic mouse gliomas and quantitation. (G) Correlation between the levels of fibrin polymers and CD163+ TAMs in the brain sections of tumor-bearing mice with or without RT. r value determined by Pearson's correlation coefficient analysis. Results are presented as mean±SD. * p<0.001.

FIG. 4. F3 knockdown suppresses radiation-induced coagulation, SASP factor secretion and TAM activation. (A) IHC images of fibrin, IBA1, and CD44 in the brains of 827 tumors expressing either non-targeting (NT) or F3 shRNAs (KD) with or without RT. (B) Kaplan-Meier survival curves of mice in (A). n=8 for each group. p<0.001 by log-rank analysis. (C) Immunoblots to determine the activation status of NFκB and STAT3, and integrin in F3 KD GBM cells with or without RT. (D) Luciferase reporter assays to measure transcriptional activities of NFκB and STAT3 signaling. (E) Survival of irradiated F3 KD cells with forced activation of STAT3 or NFκB. (F) Levels of the secreted proteins from GBM cells with or without F3 KD and RT. (G) Recruitment of macrophage (MMφ-like U937 cells co-cultured with the above cell groups. (H and I) Levels of cytokine secretion in the cells with forced activation of STAT3 or NFκB (H) or inhibition of NFκB, STAT3, integrin signaling (I). Details of inhibitors are in Method section. SASP factor index is an arbitrary unit calculated from total amounts of the secreted proteins. (J) IHC images of doxycycline-inducible F3 shRNA expressing tumors. Tumors were harvested 7 days after doxycycline treatment.

FIG. 5. ΔFVII treatment impeded RT-induced coagulation, SASP factor secretion, and TAM accumulation in vivo. (A) Schematic of F7 deletion mutant structures. (B) Co-immunoprecipitation (IP)-immunoblots of F3 and Ubiquitin (Ub) in naïve and irradiated GBM cells treated with ΔFVII recombinant protein for 1 day. Levels of ubiquitinated F3 proteins were quantitated by densitometry. (C to E) Immunoblots of F3 (C), pp65 and pSTAT3 (D), and integrin signaling components (E) in irradiated GBM cells treated with ΔFVII for 1 day. (E) Co-IP blots of F3-integrin β1 (ITGB1) immunocomplexes. (F) Cell survival of normal brain cells and irradiated GBM cells treated with ΔFVII. Irradiated GBM cells, NPCs, and astrocytes were cultured with various concentrations of ΔFVII for 3 days and cell survival was determined by MTT assay. (G) Live-cell imaging of RT-F3⁺ 022 GBM cells treated with ΔFVII. Cell growth was monitored in real-time over 12 days. (H) Quantitation of the secreted proteins using the conditioned media from irradiated GBM (022 and 827) cells treated with ΔFVII for 1 day. (I) Representative staining images of fibrin, CD163, CCR2, and cleaved-caspase-3 (C-cas3) in the 827 GBM-derived PDX tumors treated with RT, ΔFVII, or both. (J) Immunoblots of F3, pSTAT3, BBC3, C-cas3, bFGF, HGF, and H3K9me3 proteins using GBM tumor lysates. (K) Levels of the secreted proteins from the above tumor sets. (L) Tumor sizes and fibrin staining in the above tumor sets harvested 5 days after RT. (M) Tail bleeding times of tumor-bearing mice treated with RT, ΔFVII, or both. n=3 for each group. (N) Tumor volumes of the above groups (n=6). Results are presented as mean±SD. * p<0.001.

FIG. 6. ΔFVII therapies radio-sensitize GBM tumors in orthotopic PDX models. (A) IHC images of fibrin, CD44, and CD163 in orthotopic 827 GBM tumor-bearing mice treated with RT, ΔFVII, or both. Recombinant ΔFVII protein (50 μg/kg body weight) was administered via intravenous injection daily, concurrent with irradiation. (B and C) Representative immunoblots of Fibrin, FN1, YKL-40, HUTS4, pp65, CD163, and CD206 in GBM tumors (B) and quantitation in (C). (D) IF images of fibrin/FN1, CD44, CD163, and C-cas3 and quantitation. (E) H&E staining images of the mouse brain sections from combination treated group. Human tumor cells were stained with STEM121 antibody (human cell-specific marker). (F and G) D-dimer levels in plasma (F) and tail bleeding time determination (G). N=3 for each group. (H) Kaplan-Meier survival curves of tumor bearing mice treated with radiation, ΔFVII, or both. n=10 for each group. Combination-treated group showed a significant survival extension, compared to all other groups. p<0.001 by log-rank analysis.

FIG. 7. Expression of senescence and coagulation gene sets in matched primary and recurrent human GBM pairs. (A) Heatmap plots of the senescence, coagulation, NFκB, cell cycling, and stemness gene set expression in early (n=11) and late relapsed (n=14) GBM pairs. (B) Quantitation of each gene signature expression in the following tumor sets. EP, early-relapse GBM patients' primary tumors; ER, early-relapse GBM patients' recurrent tumors; LP, late-relapse GBM patients' primary tumors; LR, late-relapse GBM patients' recurrent tumors. * p<0.001. (C and D) Scatter plot showing the correlations of coagulation and senescence signature gene expression (C), and NFκB and coagulation (D). r values were determined by Pearson's correlation coefficient analysis. (E) Schematic illustration to depict the roles of F3 signaling in RT-induced GBM remodeling.

FIG. 8. RT-induced SA-βGal+ cells in GBM and normal fibroblasts, related to FIG. 1 (A) Experimental design to interrogate the biology of irradiation-induced SA-βGal+ GBM cells. (B and C) Images of SA-βGal activity in patient GBM tissue slices (B) and GBM organoids (C). Freshly resected brain slices from GBM patients and tumor-derived organoids were not irradiated or irradiated and stained with X-gal or C₁₂FDG. Percentages of C₁₂FDG⁺ cells were quantitated. Nuclei were labeled with DAPI staining (blue). (D) t-SNE plots of IMR-90 cells from RT timecourse experiments. A color gradient to depict the scores of cell cycling gene signature in each cell (total of 34,904 cells) is shown. (E) Representative images of SA-βGal staining of IMR-90 cells after a RT timecourse. C₁₂FDG⁺ cells in RT time course using two different normal fibroblast cell lines were quantitated. (F) t-SNE plots of GBM single cells in the sorted C₁₂FDG⁺ GBM cells were analyzed (total of 105,653 cells). C₁₂FDG⁺ GBM cells were sorted 5 days after radiation. (G) Heatmap plots of cell cycling gene signature in RT-C₁₂FDG⁺ and C₁₂FDG⁻ GBM cells. (H) Immunoblots of PCNA in three matched sets of RT-C₁₂FDG⁺ and C₁₂FDG⁻ GBM cells. Tubulin was used as a loading control. Results are presented as mean±SD. * p<0.001.

FIG. 9. RT-induced F3 signaling in GBM, related to FIG. 2 (A and B) Representative flow cytometry plot of F3 and ABCG2 proteins in the sorted RT-C₁₂FDG⁺ and C₁₂FDG⁻ GBM cells (A) and immunoblots (B). (A) Black line indicates staining with a control antibody. (C) IF staining images of C₁₂FDG and F3 in GBM tissue slices. (D) Levels of C₁₂FDG⁺ cells 5 days after radiation (5 or 10 Gy) using 5 different patient-derived GBM cells. (E) Levels of F3⁺ cells in 9 different sets of naïve and RT GBM cells. (F) Levels of annexin V⁺ cells in RT-F3⁺ and F3⁻ GBM cells, as determined by flow cytometry. % of annexin V⁺ cells in naïve condition was set to 1. (G) SA-βGal and F3 staining in whole-mount GBM organoids. High-magnification images of F3 staining in the core and the edge regions are shown (marked as C and E boxes, respectively). (H) IF staining images of F3 and representative markers for stem cell-associated pathway activation (Sox2, NFκB and WNT) in GBM organoids. (I) Enriched transcription factor-binding motifs in irradiated C₁₂FDG⁺ GBM cells. (J) t-SNE plots of 827 GBM single cells from RT timecourse experiments. Mono-color gradient was overlaid with mRNA levels of STAT3, CD44, and CD109 in single cells. (K) Co-IF images of F3 and stem cell-associated pathway activation in patient GBM slices. Results are presented as mean±SD. * p<0.001.

FIG. 10. RT-induced changes in GBM and TME, related to FIG. 3. (A) IF staining images of CD42b (a marker for activated platelet) and Ci-H3 (a marker for neutrophil extracellular traps (NETs)) within the irradiated PDX tumors. Numbers of CD42b⁺ cells and Ci-H3 spots were counted in 5 random fields. (B) Representative IF images of fibrinogen, fibrin, FN1, F3, IBA1, and CD163 in GBM tissue slices (432, 349, and 4674) with or without ex vivo irradiation. (C) Heatmap plots of the SASP factors in naïve or ex vivo irradiated GBM tissue slices (n=4). Conditioned media from GBM slices with or without irradiation were collected and processed to determine the levels of chemokines/cytokines (n=120). Representative SASP factors are shown below. Results are mean±SD. * p<0.001. NS, not significant.

FIG. 11. Downstream signaling of RT-induced F3 signaling in GBM, related to FIG. 4 (A) Immunoblots of F3 in GBM cells expressing either non-targeting (NT) or two independent F3 shRNAs (KD1 or KD2). Transcriptional activities of NFκB and STAT3 signaling in F3 KD GBM cells were determined by luciferase reporter assays. (B) Immunoblots of active-βcatenin and YKL-40 in F3 KD GBM cells with or without RT. (C) Heatmap plots of the secreted SASP factors in F3 KD GBM cells with or without RT. Conditioned media from GBM cells with or without irradiation were collected. (D) Immunoblots of pSTAT3, pp65, pIκBα, and HUTS4 in lysates from control and F3-overexpressing (O/E) GBM cells. (E) Real-time cell growth kinetics of F3-overexpressing (O/E) GBM cells cultured in the absence of EGF and FGF2. (F) The levels of SASP factor index in F3-O/E GBM cells. Results are mean±SD. * p<0.001.

FIG. 12. ΔFVII recombinant protein as a potent F3 targeting agent, related to FIG. 5. (A) Coagulation activities of WT FVII, and S494A (protease-inactive) FVII mutant, and ΔFVII recombinant proteins, as determined by in vitro coagulation assays. (B) Quantitation of HUTS4 levels in GBM cells treated with or without radiation and ΛFVII. Values of naïve cells are set to 1. (C) Immunoblots of pFAK and pERK in lysates from NPCs treated with ΔFVII for 1 day. (D) Immunoblots of BBC3 proteins in GBM cells treated with ΔFVII, irradiation, or both. RT alone did not significantly induce BBC, but BBC3 (PUMA) was upregulated after ΔFVII treatment. (E) Representative flow cytometry plots to measure mitochondrial membrane potential in irradiated GBM cells with or without ΔFVII. Lack of TMRE dye staining (marked in red boxes) indicates disruption of mitochondrial membrane. (F) Ultrastructural morphologies of ΔFVII-treated GBM cells with or without RT, as visualized by electron microscopy. Inset shows disrupted mitochondrial structure. (G) Images of SA-βGal activity in tumors stained with X-gal or C₁₂FDG. The numbers of SA-βGal⁺ cells were quantitated (4 animals per each). (H) Clonogenic growth of RT-C₁₂FDG⁺ subpopulations derived from subcutaneous tumors with or without ΔFVII. (I) Quantitation and heatmap plots of cytokines and chemokines in FIG. 5K. See also FIG. 5.

FIG. 13. ΔFVII therapy via neural progenitor cell carrier impedes tumor growth and GBM radio-resistance in orthotopic PDX models, related to FIG. 6 (A) IF images of human ΔFVII protein in the brains of 827 tumor-bearing mice treated with ΔFVII protein. Brains were harvested 3 hours after intravenous injection of recombinant ΔFVII protein (50 μg/kg body weight). (B) Co-culture experiments with ΔFVII-expressing NPCs. ΔFVII-transduced NPCs were co-cultured with irradiated GBM cells, NPCs, or astrocytes in transwell plates. ΔFVII protein secreted into the conditioned media (supernatant) was determined by immunoblots. Two days after co-culture, annexin V assays were performed on 022 RT GBM cells and NPC/astrocytes. (C) IF images of NPC-based cell carrier for ΔFVII therapy. Control or ΔFVII-expressing NPCs were injected into the brains of 827 tumor-bearing mice 16 days after tumor cell implantation, followed by in vivo irradiation two days later. Quantitation of F3⁺, CD163⁺, or C-cas3⁺ cells in the brain sections is shown (n=3 per each group). (D) Representative IF images of F3, TS2/16, CD44, Nestin, and pβCAT in the brain sections from ΔFVII-NPC experiments. (E) Kaplan-Meier survival curves of each group (n=8). Combination-treated group showed a significant survival extension, compared to all other groups. p<0.001 by log-rank analysis.

FIG. 14. RT-induced F3 signaling can be applicable to other therapy models and cancer types, related to FIG. 7. (A) Kaplan-Meier survival curves of each group (n=8). TMZ (25 mg/kg body weight) were administered via intravenous injection, concurrent with irradiation. Triple combination-treated group showed a far greater survival extension, compared to all other groups. P<0.001 by log-rank analysis. (B) Immunoblots of F3 protein in lysates from patient-derived GBM cells (131, 559, 592, 827) treated with kinase inhibitors (PHA 665752 (c-MET inhibitor), 2 μM; gefitinib (EGFR inhibitor), 5 μM; and BYL 719 (AKT inhibitor), 1 μM). (C) Top; staining images of Cleaved-Caspase-3 (C-cas3) in 131 GBM cells two days after the indicated treatments. Scale bars, 20 μm. Bottom; quantitation of C-cas3⁺ cells counted in 5 random fields (left) and limiting-dilution assay to determine clonogenic growth of 131 GBM cells treated with PHA665752, ΔFVII (100 ng/ml), or both (right). (D) Representative staining images for senescence-associated markers (SA-βGal and LaminB1) in MG63 cells. MG63 cells were irradiated with 3 Gy, harvested at the indicated time points, and processed for staining analyses. Scale Bar, 20 m. (E) Immunoblots of F3, LaminB1, pp65, and pGSK3β in a RT time course of MG63 cells. (F) IHC images of fibrin in the lung sections. Naïve and irradiated MG63 cells were injected into nude mice through tail vein, and mice were killed 2 weeks later. Arrows mark bronchiolar epithelium. Arrowhead indicates tumor cell cluster. Scale Bar, 200 m. (G) Quantitation of senescence, SASP, and NFκB signatures in naïve and irradiated MG63 single cells (total 37,531 cells) (H) t-SNE plots of MG63 single cells in a RT time course, overlaid with mRNA levels of CCL2 and FN1 in each cell (total 37,531 cells).

FIG. 15 provides the amino acid sequence of human Factor VII (SEQ ID NO:1), and the locations of the various domains of this protein.

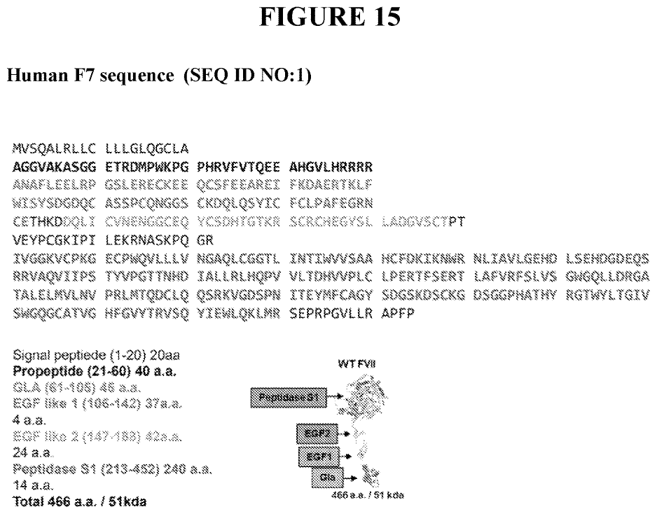

FIG. 16 provides the alignment of the domains of human Factor VII and various deletion constructs.

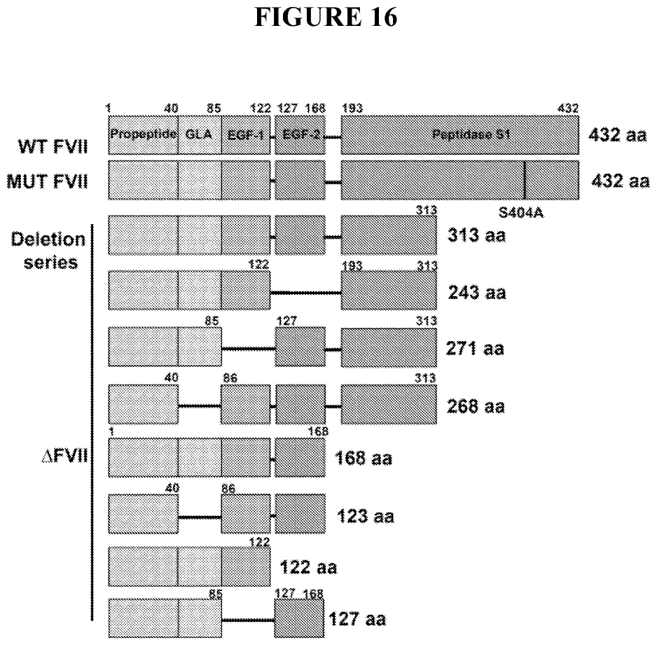

FIG. 17 shows the results of cell killing activity of various human delta Factor VII constructs.

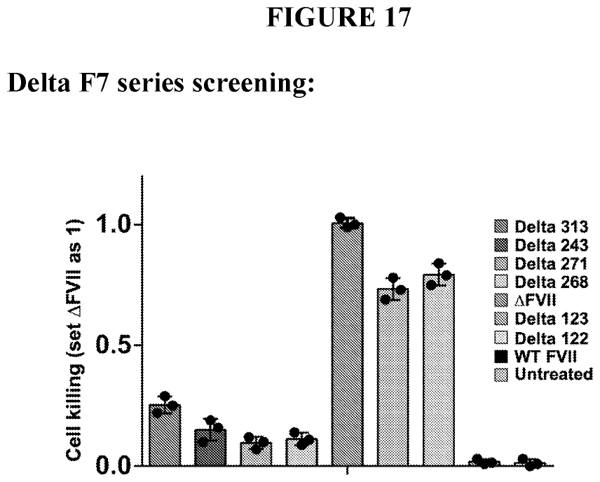

FIG. 18 shows a human delta F7 recombinant construct with 13 amino acid Tev Protease recognition sequence and his tag (SEQ ID NO:2)

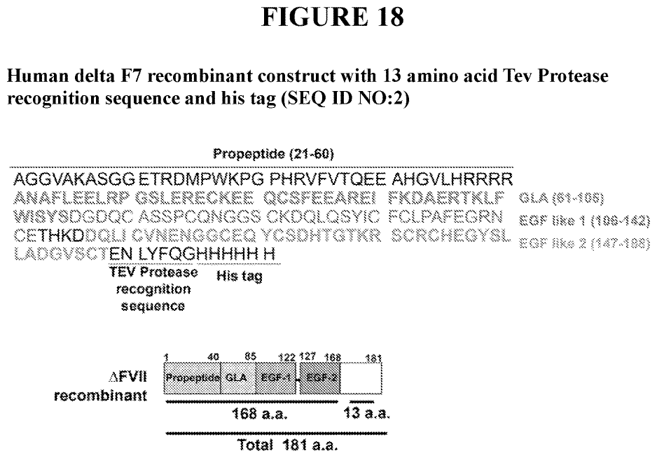

FIG. 19A shows the delta FVII Construct Amino Acid Sequence (SEQ ID NO:3) and graphic of construct. FIG. 19B shows the delta 123 Construct Amino Acid Sequence (SEQ ID NO:4) and graphic of construct. FIG. 19C shows the delta 122 Construct Amino Acid Sequence (SEQ ID NO:5) and graphic of construct.

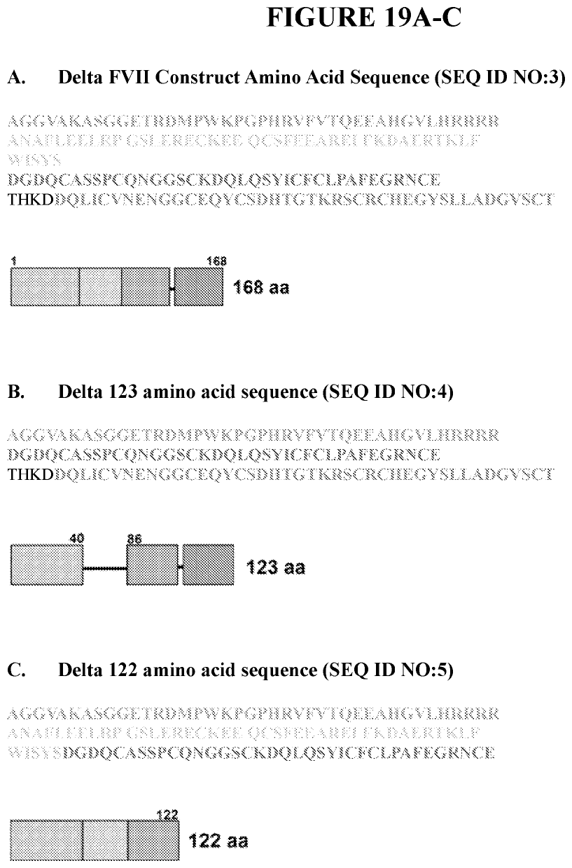

FIG. 20 shows a ΔFVII construct (amino acid sequence SEQ ID NO:3) rapidly increased serine phosphorylation in the cytosolic domain of F3 protein, and promoted ubiquitination, mimicking molecular changes shown in the natural F3 degradation pathway. Also, ΔFVII inhibited interaction between F3 and integrin, which is an important event for integrin signaling activation. Its effect was ultimately validated in various GBM cells, all of which showed dramatic decreases in F3 levels.

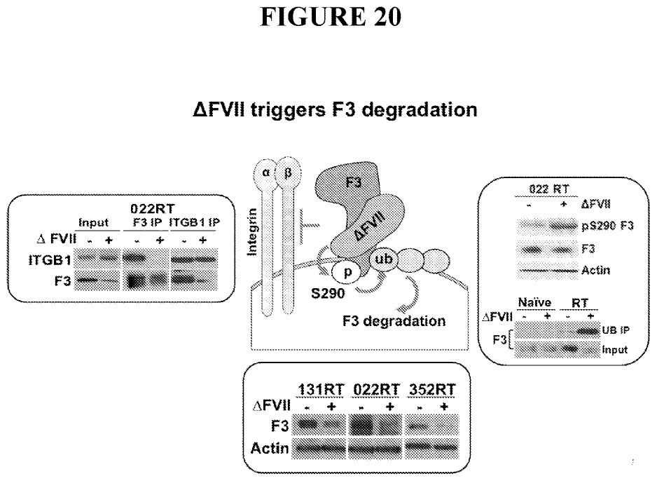

FIG. 21 shows a ΔFVII construct (amino acid sequence SEQ ID NO:3) therapy radio-sensitizes BM tumors in orthotopic brain metastasis (BM) PDX models. Kaplan-Meier survival curves of tumor bearing mice treated with radiation, ΔFVII, or both. n=7 for each group. Combination-treated group showed a significant survival extension, compared to all other groups. p<0.001 by log-rank analysis.

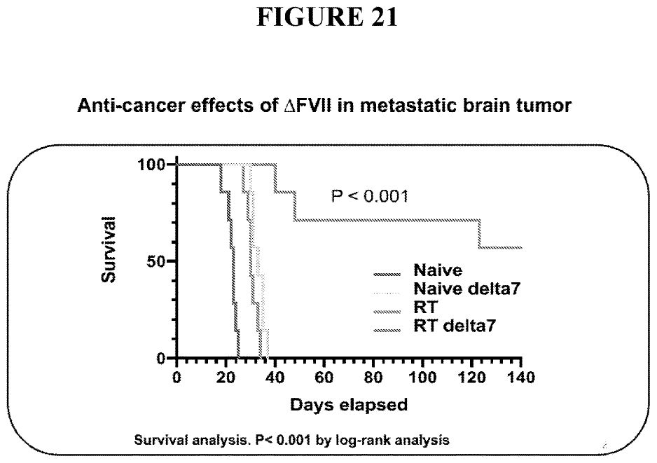

### DEFINITIONS

To facilitate an understanding of the invention, a number of terms are defined below.

As used herein, the term “codon” or “triplet” refers to a group of three adjacent nucleotides which specify one of the naturally occurring amino acids found in polypeptides. The term also includes codons which do not specify any amino acid. It is also noted that, due to the degeneracy of the genetic code, there are many codons that code for the same amino acid. As such, many of the bases of the nucleic acid sequences of the present invention can be changed without changing the actual amino acid sequence that is encoded. The present disclosure is intended to encompass all such nucleic acid sequences.

As used herein, the terms “an oligonucleotide having a nucleotide sequence encoding a polypeptide,” “polynucleotide having a nucleotide sequence encoding a polypeptide,” and “nucleic acid sequence encoding a peptide” means a nucleic acid sequence comprising the coding region of a particular polypeptide. The coding region may be, for example, present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc., or a combination of both endogenous and exogenous control elements.

Also, as used herein, there is no size limit or size distinction between the terms “oligonucleotide” and “polynucleotide.” Both terms simply refer to molecules composed of nucleotides. Likewise, there is no size distinction between the terms “peptide” and “polypeptide.” Both terms simply refer to molecules composed of amino acid residues.

As used herein, the term “the complement of” a given sequence is used in reference to the sequence that is completely complementary to the sequence over its entire length. For example, the sequence 5′-A-G-T-A-3′ is “the complement” of the sequence 3′-T-C-A-T-5′.

The term “isolated” when used in relation to a nucleic acid, as in “an isolated oligonucleotide” or “isolated polynucleotide” or “isolated nucleic acid sequence encoding an aggregated Tau binding molecule” refers to a nucleic acid sequence that is identified and separated from at least one contaminant nucleic acid with which it is ordinarily associated (e.g. host cell proteins).

As used herein, the term “purified” or “to purify” refers to the removal of contaminants from a sample. For example, delta VII constructs herein may be purified by removal of contaminating proteins. In another example, recombinant delta VII constructs herein are expressed in bacterial host cells and the polypeptides are purified by the removal of host cell proteins. The percentage of recombinant delta VII constructs is thereby increased in the sample.

### DESCRIPTION OF THE INVENTION

Provided herein are compositions, kits, and methods for treating a human subject with cancer (e.g., brain cancer) with a composition comprising: a human delta Factor VII construct, or nucleic acid sequence encoding said human delta Factor VII construct. In certain embodiments, the human delta Factor VII construct comprises a Propeptide domain and EGF-1 domain, or portions thereof, and lacks a functioning Peptidase S1 domain. In some embodiments, the human subject has glioblastoma and has been previously treated with radiation therapy.

In work conducted during development of embodiments herein, we used an integrative approach to understand tumor cell senescence in the course of radiation resistance, combining the assays with a modified SA-βGal substrate that allows sorting and live cell fate tracing of the senescent tumor cells, cell surface marker screening, single cell RNA sequencing, and profiling of transcriptome and chromatin landscapes. We demonstrate a causal link between RT-induced senescence and global oncogenic reprogramming and derive therapeutic strategies to inhibit these processes using the delta factor VII constructs herein. In certain embodiments, the various segments of human Factor VII have the amino acid sequences as shown below:

Human Factor VII Propeptide:
(SEQ ID NO: 6)
AGGVAKASGGETRDMPWKPGPHRVFVTQEEAHGVLHRRRR
Human Factor VII GLA:
(SEQ ID NO: 7)
ANAFLEELRPGSLERECKEEQCSFEEAREIFKDAERTKLFWISYS
Human Factor VII EGF-1:
(SEQ ID NO: 8)
DGDQCASSPCQNGGSCKDQLQSYICFCLPAFEGRNCE
Human Factor VII EGF-2:
(SEQ ID NO: 9)
DQLICVNENGGCEQYCSDHTGTKRSCRCHEGYSLLADGVSCT

The human delta Factor VII constructs herein can be administered by any suitable methods (e.g., intravenous, intracranial, oral, etc.) and may formulated with a physiologically acceptable carrier or excipient to prepare a pharmaceutical composition. The carrier and composition can be sterile. The formulation should suit the mode of administration. Suitable pharmaceutically acceptable carriers include but are not limited to: water, salt solutions (e.g., NaCl), saline, buffered saline, alcohols, glycerol, glucose, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances, and the like that do not deleteriously react with the active compounds. The compositions containing the human delta Factor VII constructs, or nucleic acid encoding such constructs (e.g., DNA or mRNA) may be packages into suitable carriers such as liposomes, nanoparticles, gel caps, etc.

### EXAMPLES

The following examples are provided in order to demonstrate and further illustrate certain preferred embodiments and aspects of the present invention and are not to be construed as limiting the scope thereof.

### Example 1

### Tissue Factor is a Critical Regulator of Radiation Therapy-Induced Glioblastoma Remodeling

Radiation therapy (RT) provides therapeutic benefit for patients with glioblastoma (GBM), but inevitably induces poorly-understood global changes in GBM and its microenvironment (TME) that promote radio-resistance and recurrence. Through a cell surface marker screen, we identified that CD142 (Tissue factor or F3) is robustly induced in the senescence-associated β-galactosidase (SA-βGal)-positive GBM cells after irradiation. F3 promotes clonal expansion of irradiated SA-βGal+ GBM cells and orchestrates oncogenic TME remodeling by activating both tumor-autonomous signaling and extrinsic coagulation pathways. Intratumoral F3 signaling induces a mesenchymal-like cell state transition and elevated chemokine secretion. Simultaneously, F3-mediated focal hypercoagulation states lead to activation of tumor-associated macrophages (TAMs) and extracellular matrix (ECM) remodeling. F3-targeting agents potently inhibits the above oncogenic events and impedes tumor relapse in vivo. These findings support F3 as a critical regulator for therapeutic resistance and oncogenic senescence in GBM.

### Methods

### Patient-Derived Glioblastoma Specimens and Derivatives

Following written informed consent, tumor samples classified as GBM, based on the WHO criteria, were obtained from patients undergoing surgical treatment in accordance with the NIH, Cleveland Clinic Lerner Research Institute, and Samsung Medical Center Institutional Review Boards. Within 1 to 6 hours after surgical removal, tumors were washed in PBS and processed for the following models.

GBM spheroids: Tumor cells were cultured in Neurobasal medium supplemented with N2, B27 and bFGF and EGF (NBE medium; Neurobasal media, N2 and B27 supplements (0.5× each; Invitrogen), and human recombinant bFGF and EGF (25 ng/mL each; R&D Systems)^{1,2}.

GBM organoids: GBM organoids display tumor cell hierarchy and differentiation heterogeneity by oxygen/nutrient gradient. Tissue pieces were cut into 0.5 to 1 mm diameter and cultured in the dishes on top of an orbital shaker rotating at 120 rpm at 37° C. in a 5% CO₂, 95% humidity incubator³⁻⁵.

GBM slices: GBM tissue pieces were sliced using a vibrating blade microtome into 3 to 5 mm diameter, 300 μm thick slices and transferred to a culture dish.

### Primary Cell Cultures

Normal NPCs derived from human embryonic stem cells (H9, Invitrogen, Cat #N7800100) and aborted fetal brain tissues (Lonza, Cat #PT-2599) were cultured in NBE media or neural progenitor maintenance media (Lonza, Cat #CC-3209). Primary human astrocytes (Lonza, Cat #CC-2565) were cultured in astrocyte growth media (Lonza, Cat #CC-3186). Human U937 cells from ATCC (CRL-1593.2) were maintained in RPMI 1640 media (Gibco,11875093) with 10% FBS (Gibco,10438026), and 1% penicillin/streptomycin (Gibco, 15140148). For all co-culture experiments, FBS serum was not used.

### Patient-Derived Xenograft (PDX) and RCAS-TVA Mouse Glioma Models

All mice experiments were performed according with the IACUC approved protocols. For orthotopic tumor implantation, GBM cells were injected intracranially into the striatum of nude mice (BALB/c nu/nu) by using a stereotactic device (Kopf instruments, coordinates: 2 mm anterior, 2 mm lateral from the bregma, 2.5 mm depth from the dura) as previously described². For F3 inducible knockdown, drinking water containing doxycycline (D9891, Sigma, 2 mg/ml) and 5% sucrose was given at 20 days after injection. The water was protected from light and exchanged every 2 days. Syngeneic RCAS-TVA mouse gliomas induced by PDGFB overexpression in p53-null background were generated as previously reported⁶. When mice develop neurological symptoms (lethargy, ataxia, and seizures) or significant body weight loss, mice were killed and processed for histological analysis.

### Lung Metastasis Model

Naïve or irradiated MG63 cells (5×10⁵) were injected into the tail vein of each nude mice (BALB/c nu/nu).

### ΔFVII-Mediated Therapy in Animal Models

All mice were randomly assigned to appropriate treatment groups. For recombinant ΔFVII protein experiments, ΔFVII proteins (50 μg/kg body weight) were administered via intravenous injection daily, concurrent with irradiation or temozolomide treatment (25 mg/kg body weight, intraperitoneal injection, T2577, Sigma)⁷. For ΔFVII-expressing NPC experiments, NPCs (1×10⁶ cells per mice) were injected into the brains of tumor-bearing mice, two days before irradiation.

### Blood Clotting Time Test

To determine tail-bleeding time, mice were kept under anesthesia and placed on a heating pad. Distal tail was cut at 5 mm from the tip and immediately submerged into 10 ml PBS at 37° C.⁸.

### D-Dimer ELISA Assays

Plasma was collected in 3.2% buffered sodium citrate tube from each sample for D-dimer detection. The amount of D-dimer was assayed using mouse D-dimer ELISA kit (Novus Biologicals, NBP3-08100) according to manufacturer's protocol.

### Radiation Regime

For irradiation of tissues and cells, a single dose of 3 Gy, 5 Gy, or 10 Gy was used. For in vivo irradiation, the anesthetized mice were placed in a lead shielding device in which the brains or subcutaneous tumors were exposed. Localized radiation was performed with either a single dose of 10 Gy or a fractionated regime (2 Gy daily for 5 days).

### Senescence-Associated β-Galactosidase (SA-βGal) Reactivity Assays

For colorimetric SA-βGal assay, we used senescence-β-galactosidase kit (9860, Cell Signaling). Briefly, samples were incubated with β-galactosidase staining solution (pH 6.0) at 37° C. in a dry incubator without the added CO₂. Staining images were analyzed using an inverted fluorescence microscope (DM4000 B, Leica). For fluorometric SA-βGal staining assay, live cells or tissues were pre-treated with bafilomycin A1 (B1793, Sigma-Aldrich, 100 nM) and then cultured with C₁₂FDG (5-Dodecanoylaminofluorescein Di-β-D-Galactopyranoside, D2893, Invitrogen) as described with minor modifications⁹. C₁₂FDG intensity was analyzed by LSR II Fortessa flow cytometer (BD) or confocal microscope.

### Fluorescence Activated Cell Sorting (FACS)

Cell sorting was performed using BD FACS Aria II. GBM cells were stained with either C₁₂FDG or F3 antibody (BD 550312 1:20), and each subpopulation was sorted based on the levels of staining intensities. A matched isotype antibody was used as a control and propidium iodide (PI, 5 μg/ml) was used for live/dead cell determination. To ensure purity and viability of the sorted subpopulations, we repeated flow cytometry analysis and PI staining after initial sorting. Data were collected and analyzed using FlowJo software.

### Flow Cytometry Analysis

Dissociated GBM cells were incubated with 5% donkey or goat serum, 2 mM EDTA in PBS for 30 minutes to block non-specific binding, and then labeled with anti-F3-FITC antibody (BD 550312, 1:20), Nestin (SC-23927, 1:100), H3K9me3 (ab8898, 1:100), or ABCG2 (BD562167, 1:100) in 5% serum containing PBS for 1 hour. For detection of intracellular proteins, cells were permeabilized with 0.1% saponin (S7900, Sigma-Aldrich). After gently wash by cold PBS, the cells then incubated with Alexa Flour secondary antibodies (Invitrogen, 1:400) for additional 30 minutes.

Annexin V staining and mitochondria TMRE assays were performed using standard detection kits (ab113852, Abcam). Flow cytometry assays were performed using at least three independent biological samples.

### In Vivo Clonal Analysis Using Barcode-Sequencing

GBM cells were transduced with Clone Tracker 50M lentiviral barcode library (BC13X13V, Cellecta Inc.). For in vivo clonal analysis, transduced GBM cells were injected into the brains of nude mice. Genomic DNA was extracted from the resultant tumor tissues using QIAamp DNeasy Blood and Tissue kit (69504, Qiagen) following manufacturer instructions. Barcodes from the tumor were amplified using sample-specific primer sets provided in the NGS prep kit (LNGS-200, Cellecta Inc.). Library quality and fragment sizes were assessed on a Fragment Analyzer before high-throughput sequencing on a HiSeq. Sequence processing and analysis were performed by using Cellecta NGS Demultiplexing and Alignment software.

### Cell Surface Marker Screening

BD lyoplate human cell surface marker screening panel contains 242 purified monoclonal antibodies against human clusters of differentiation (CD) markers (560747, BD). Patient-derived GBM cells (131 and 559) were used for cell surface marker screening. Briefly, GBM cells (about 1.5×10⁸) were dissociated with Accutase (A6964, Sigma-Aldrich) and incubated with bafilomycin A1 (100 nM) and C₁₂FDG (33 μM) for 2 hours at 37° C. or anti-F3-FITC for 1 hour at 4° C. After C₁₂FDG or F3 staining, cells were split and incubated with each CD antibody for 1 hour at 4° C. Flow cytometry data were measured using LSRII HTS system and analyzed using FlowJo software (NIH).

Single Cell RNA-Sequencing (scRNA-Seq) and Bulk RNA-Seq Analysis

Cells were dissociated with Accutase and suspended in 1% BSA PBS solution. Live cell FACS sorting was performed with DRAQ5 fluorescent probe (62251, Thermo Fisher Scientific). Cell vitality was determined by trypan blue staining and live cells were diluted to a final concentration of 1000 viable cells/μL in 0.1% BSA PBS solution. Each sample had over 90% viability. ScRNA-seq library preparation and sequencing were performed as previously reported^{3,10}. Briefly, scRNA-seq data were processed through 10× Genomics Chromium Single Cell Platform, and count matrices were generated using their Cell Ranger pipeline (10× Genomics). ScRNA-seq data were analyzed using scanpy. For quality control, genes detected in less than 5 cells and cells with fewer than 1000 genes were excluded. Expression values were corrected to 100,000 reads per cell and transformed. Unbiased clustering was performed by UMAP dimensionality reduction visualization analysis¹⁰. Gene signature sets used in this report are; GBM subtype¹¹, stemness^{12,13}, cell cycling¹⁰, NFκB¹⁴, senescence¹⁵⁻¹⁷ and coagulation¹⁸.

### ATAC-Sequencing Analysis

Cells were stained with either C₁₂FDG or F3 antibody (BD 550312, 1:20), and each subpopulation was sorted using BD FACS Aria II. Propidium iodide (PI, 5 μg/ml) was used for live/dead cell determination. 5×10⁵ cells/sample were used for ATAC-Seq Library Preparation (K1157, APExBio). Briefly, cells were resuspended in 50 μl ATAC lysis buffer containing (0.5 μl 10% NP40, 0.5 μl 10% Tween 20, and 0.5 μl 2% Digitonin) for 3 min on ice and then cold ATAC lysis buffer containing (0.5 μl 10% Tween 20) was added. Nuclei were centrifuged at 1000 rpm for 10 min at 4° C. in a fixed angle. Nuclei were resuspended in 50 μl tagmentation master mix containing (5 μl transposase, 16.5 μl PBS, 0.5 μl 2% Digitonin, 0.5 μl 10% Tween 20, 2.5 μl water, and 25 d 2× tagmentation buffer). The tagmentation reaction was incubated at 37° C. for 30 min in a thermomixer with 1000 rpm. Reactions were cleaned up with DNA purification kit (28104, Qiagen). Libraries were amplified and sequenced on a Nextseq instrument (Illumina).

### Bioinformatics Data Analysis

Analysis for genomic alterations including amplification, deletion, and mutation of key genes and glioma subtype assignment were performed, as described^{11,19-22}. Pearson correlation coefficient was calculated by “cor” function of R and Pearson's Chi-squared Test was conducted using “chisq.test” function of R with default settings.

### Chemokine/Cytokine Profiling Assays

Conditioned media was filtered through 0.2 m filters (Sartorious Stedium Biotech). Filtered media or cell lysates were incubated with Human Cytokine antibody array kit (AAH-CYT-1000, RayBiotech). Intensity of each spot was measured using ImageJ and analyzed using the RayBiotech analysis tool (AAH-ANG-1000, RayBiotech). Heatmaps were generated by “heatmap.2” function of R package.

### Lentivirus

Human HEK293 cells (ATCC) were cultured in DMEM media supplemented with 10% FBS, 1% penicillin and streptomycin. For viral production, 293T cells were co-transfected with the corresponding lentiviral vector and packaging plasmids (psPAX2 and pCMV-VSV-G) using CalPhos Mammalian Transfection Kit (631312, Clontech). Virus-containing supernatants were collected and concentrated by Lenti-X concentrator (631231, Clontech).

All expression vectors were cloned into pLenti6/V5 vector (K495500, Invitrogen) and validated by sequencing and immunoblot analysis. Expression vectors used in this study include wildtype F3, wildtype F7, F7 deletion series, STAT3C mutant, and IKK-2 S177E S181E (IKK2SSEE) mutant (a gift from Anjana Rao, Addgene plasmid #11105).^{2,3}. A series of F7 deletion mutants was designed to include and/or exclude domains of F7 proteins. Short hairpin RNA (shRNA)-expressing lentiviral vectors were purchased from Sigma-Aldrich and doxycycline-inducible shRNA plasmids were purchased from Dharmacon. F3 or pp65 shRNA constructs were tested and at least two independent shRNA knockdown vectors were selected for further studies.

### ΔFVII Recombinant Proteins

We designed various recombinant variant FVII proteins including ΔFVII and S404A FVII mutant as well as wild type FVII²⁴. Recombinant FVII variant proteins including wildtype F7, S404A F7, and ΔFVII (188 amino acid protein without peptidase S1 domain) were synthesized (Genscript) and validated by immunoblot analysis, in vitro clotting assay (factor 7 human chromogenic activity Assay; ab108830, Abcam), and cysteine bond determination by MASS spectrometry.

### Immunofluorescence Analysis

Tissue slices were harvested 1 to 3 days after irradiation and fixed in 4% PFA (SC281692, Santa Cruz biotechnology). To prepare the frozen sections of tumors, samples were washed in PBS, cryoprotected in 30% sucrose (S0389, Sigma-Aldrich) at 4° C. overnight, embedded in O.C.T compound (4583, SAKURA Tissue Tek), and sectioned using a cryostat (CM3050S, Leica). Tissue sections and cells were blocked using a blocking solution (0.3% Triton X-100, 5% goat or donkey serum, 1% BSA in PBS) for 1 hour at room temperature. For mouse tissue sections, mouse-on-mouse blocking reagent (MKB-2213, Vector Laboratories) was added to the blocking solution. Immunofluorescence images were taken using a Leica TCS SP5 Confocal Microscope.

### Immunohistochemistry Analysis (IHC)

Paraffin sections were prepared in the Cleveland Clinic Lerner Research Institute imaging core. For immunohistochemistry analysis (IHC), tissue sections were deparaffinized in xylene (214736, Sigma-Aldrich) and rehydrated through ethanol gradient. Antigen retrieval was achieved by microwaving the sections in Unmasking solution (citrated based buffer, pH 6.0, H-3300, Vector Laboratories). Endogenous peroxidase activity was blocked by incubation with BLOXALL blocking solution (SP-6000, Vector Laboratories). All images were taken by slide scanner Leica SCN400 microscope and analyzed by Imagescope and ImageJ.

Immunoblots And co-Immunoprecipitation

Cells were lysed in Pierce IP lysis buffer (25 mM Tris-HCl pH 7.4, 150 mM NaCl, 1 mM EDTA, 1% NP-40, and 5% glycerol; #87788, Thermo) supplemented with protease inhibitors cocktail (Complete Mini, 11836153001, Sigma-Aldrich) and phosphatase inhibitor (#78428, Thermo), incubated on ice for 30 minutes and cleared by centrifugation at 4° C. for 20 minutes. For immunoprecipitation, protein lysates were incubated with appropriate antibodies for overnight at 4° C. Protein bands were visualized using ECL Western Blotting Detection Reagents (RPN2232, GE Healthcare) and subjected to densitometry analysis using ImageJ.

### Cell Growth, Viability, Drug Treatment, and Soft Agar Colony Forming Assays

Standard methods including cell counting and MTT assays (11465007001, Sigma-Aldrich) were used. Cilengitide (1 to 5 M, 56387, Selleckchem), integrin blocking antibody (ab24693, Abcam), and cucurbitacin (100 nM, C4493, Sigma-Aldrich) were used to inhibit integrin or STAT3 signaling, respectively.

Soft agar colony forming assays were performed to determine capacity of clonogenic cell growth by counting single cell-driven colonies. GBM cells were mixed with top agar (Neurobasal media, N2 and B27, 0.4% agarose) and layered on top of 0.8% agarose. Medium with fresh ΔFVII (100 ng/ml) was added every 3 days. Three weeks later, colonies were fixed with 4% PFA and stained with 0.5% crystal violet.

### Macrophage Recruitment Assay

Immune cell recruitment capacity of GBM cells and U937 cells was measured using transwell inserts (8 μm pore size, 3422). The cells were subsequently fixed with 4% paraformaldehyde at room temperature for 15 min and stained with crystal violet at room temperature for 10 min.

### NFκB, STAT3, and TCF/LEF Reporter Assays

To determine transcriptional activities of the above pathways, cells were transduced with lentiviral constructs containing NFκB consensus element, STAT3 binding elements, and TCF/LEF transcriptional response elements with the minimal promoter red firefly luciferase reporter gene (Systems Biosciences). Luciferase intensities were measured by ONE-Glo Luciferase Assay System according to manufacturer's protocol (E6110, Promega).

### Transmission Electron Microscopy (TEM)

Cells were fixed in 2.5% glutaraldehyde/4% PFA in 0.1M sodium cacodylate buffer at 4° C. overnight. Cell suspension samples were washed and treated with 1% osmium tetroxide for 1 hour, stained with 1% uranyl acetate, dehydrated, and then embedded in LX-112. Samples were analyzed using Zeiss EM 10 transmission electron microscope.

### Real-Time Live Cell Imaging Analysis

Cell growth was monitored by using Incucyte live cell analysis system (Essen Bioscience). Confluency of each well was determined every 3 hours for the entire experimental periods.

### Quantitation and Statistical Analysis

All data were expressed as means±SD from at least three independent experiments. Quantification of immuno-positive cells in immunostaining analyses was carried out using NIH imageJ software (National Institutes of Health, Bethesda, MD). For the animal survival studies, p values were determined by log-rank test. Student's t-test or ANOVA were used to determine statistical significance. Pearson correlation coefficient was calculated by “cor” function of R.

### Results

### Irradiation-Induced SA-βGal+ GBM Cells Harbor Stemness and Senescence-Like Features.

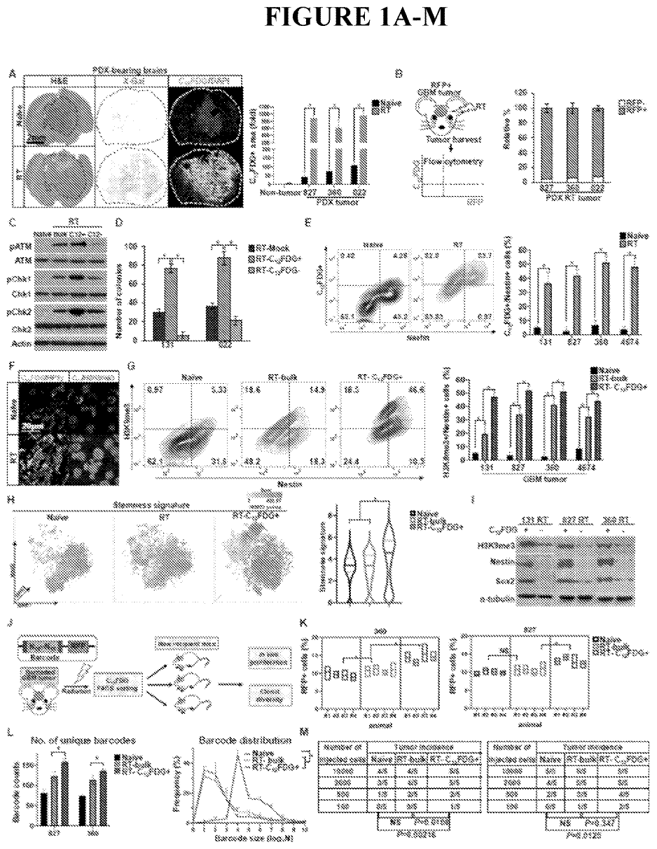

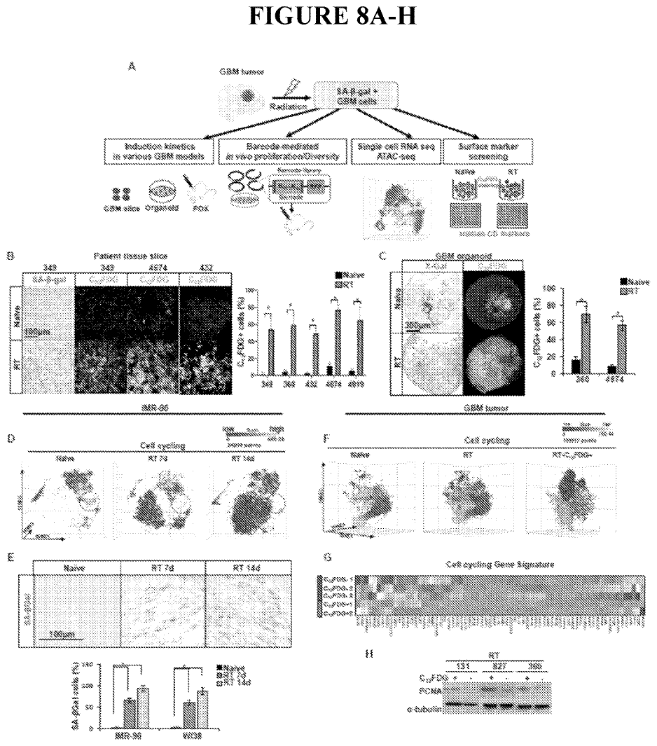

To determine the extent of senescence in GBM tumor, we utilized three different orthotopic patient-derived GBM models (FIG. 1A). 827 and 022 GBM cells have homozygous deletion of Cdkn2a, a common genomic alteration found in GBM (TCGA, 2008). The brains of tumor-bearing mice were irradiated and the activity of senescence-associated β-galactosidase (SA-βGal) were measured with either a colorimetric substrate X-Gal or a cell-permeable fluorogenic substrate C₁₂FDG (Debacq-Chainiaux et al., 2009; Dimri et al., 1995) (FIGS. 1A and 8A). Irradiation induced strong SA-βGal activity especially in the tumor regions (FIG. 1A). To determine the cell types of SA-βGal+ cells, we implanted red fluorescent protein (RFP)-transduced GBM tumors, irradiated the mice in vivo, and stained with C₁₂FDG (FIG. 1B). Flow cytometry analysis revealed that more than 90% of SA-βGal⁺ cells in the irradiated brains were positive for RFP, indicating that primarily GBM cells acquire RT-induced SA-βGal activity (FIG. 1B). To better cover inter-tumoral GBM genetic heterogeneity and to avoid model-dependent biases, we utilized multiple patient-derived GBM slices and organoids (Details are in Method sections) (Hubert et al., 2016; Jacob et al., 2020; LeBlanc et al., 2022). Across all GBM samples tested, we found robust induction of SA-βGal activity after RT (FIGS. 8 B and C).

To investigate cellular states of post-irradiated SA-βGal+ GBM cells, we separated SA-βGal⁺ and SA-βGal⁻ GBM subpopulations by C₁₂FDG staining and subsequent fluorescence-activated cell sorting (FACS). DNA damage repair activity and clonal growth capacity are critical for GBM radio-resistance and subsequent recurrence (Bao et al., 2006). C₁₂FDG⁺ cells after irradiation (RT-C₁₂FDG⁺) showed enhanced activities of ATM and DNA checkpoint kinases, relative to matched C₁₂FDG^{low/-} or mock-sorted bulk tumor cells (FIG. 1C). Furthermore, RT-C₁₂FDG⁺ cells expressed Nestin (a representative GBM stemness marker) and were highly enriched with clonogenic cells, as determined by in vitro colony forming analysis (FIGS. 1 D and E). Next, we determined the levels of representative senescence markers in GBM tumors with or without irradiation. RT-C₁₂FDG⁺ cells had high levels of HP1γ and H3K9me3 but little or no expression of CDKN2A (also known as p16, a cell-cycle arrest-associated senescence marker) (FIG. 1 F to I and data not shown). Notably, RT increased the number of GBM cells that express both Nestin and H3K9me3, which were further enriched in RT-C₁₂FDG⁺ cells (FIG. 1G).

To examine global transcriptomes of SA-βGal⁺ GBM cells, we performed single-cell RNA sequencing (scRNA-seq) and bulk RNA seq analysis. In total, we profiled about 100,000 single GBM cells and classified these cells based on the expression levels of stemness and cell cycle progression (Ben-Porath et al., 2008; Neftel et al., 2019; Suva et al., 2014). Both naïve and irradiated GBM cells contained the cell populations with high expression levels of the cell cycle and/or stemness gene signatures, reflecting their aggressive nature. Notably, RT-C₁₂FDG⁺ subpopulation harbored most of the single cells that have the highest scores for the stemness gene signature (FIGS. 1 H and I).

Cellular senescence has been extensively studied in irradiated IMR90 or WI38 cells (non-transformed human fibroblasts). Consistent with a traditional view of senescence, SA-βGal⁺ IMR90 or WI38 cells did not proliferate (FIGS. 8 D and E). In contrast, bulk RNA sequencing analysis of matched C₁₂FDG⁺ and C₁₂FDG^{low/- GBM cells after radiation showed that many of the cell cycle signature genes, including PCNA, BUB}1, and FoxM1 are highly expressed in C₁₂FDG⁺ cells (Ding et al., 2013; Zhang et al., 2011) (FIG. 8 F to H). These data suggest that SA-βGal⁺ GBM cells do not exhibit the irreversible cell cycle arrest state.

To formally test whether RT-SA-βGal⁺ GBM cells can clonally expand and contribute to post-RT tumor growth, we used the lentiviral-mediated barcode/RFP transduction technology to label GBM cells. We isolated C₁₂FDG⁺ cells from the irradiated, labeled GBM tumors and immediately injected these cells into the brains of new recipient mice (FIG. 1 J to M). RT-C₁₂FDG⁺ GBM cells generated significantly larger tumors in new recipient mice, compared to non-irradiated (naïve) or irradiated bulk tumor cells (FIG. 1K). Clonal expansion analysis by barcode sequencing revealed that larger numbers of individual barcodes were detected in the tumors derived from RT-C12FDG+ GBM cells compared to those of naïve or RT-bulk cells (FIG. 1L). This trend corroborates well with the current notion of the clonal diversity in cancer following treatment (Bhang et al., 2015; Greaves and Maley, 2012). Lastly, we performed in vivo limiting dilution tumor formation assays using the above cell populations. RT-C₁₂FDG⁺ cells derived from two different patient GBMs contained higher frequencies of tumor-forming cells than RT-bulk- or C₁₂FDG⁻ cells (FIG. 1M). Collectively, these data suggest that irradiation-induced SA-βGal⁺ GBM cells harbor sternness and some of senescence-like characteristics and that they are a cell population contributing to post-RT tumor growth.

F3, Highly Expressed in Irradiated C₁₂FDG⁺ GBM Cells, is Associated with Sternness, Cell State Transition, and an Enhanced Secretory Phenotype.

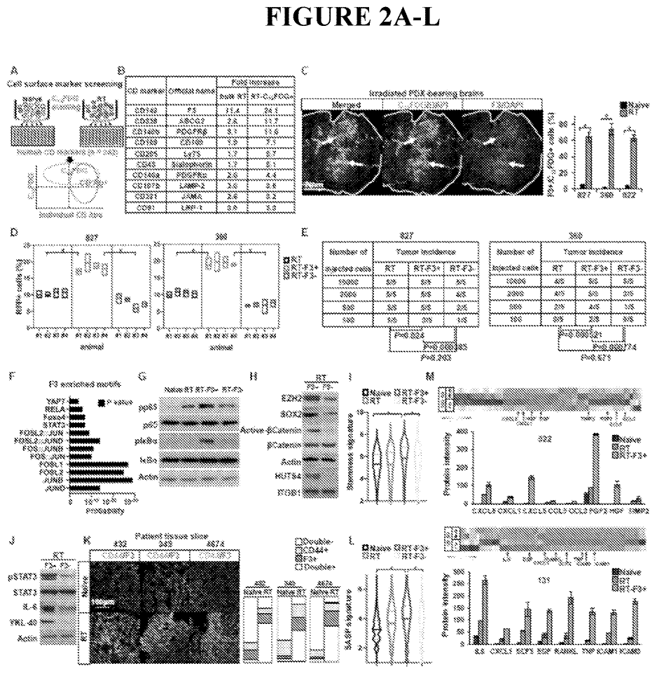

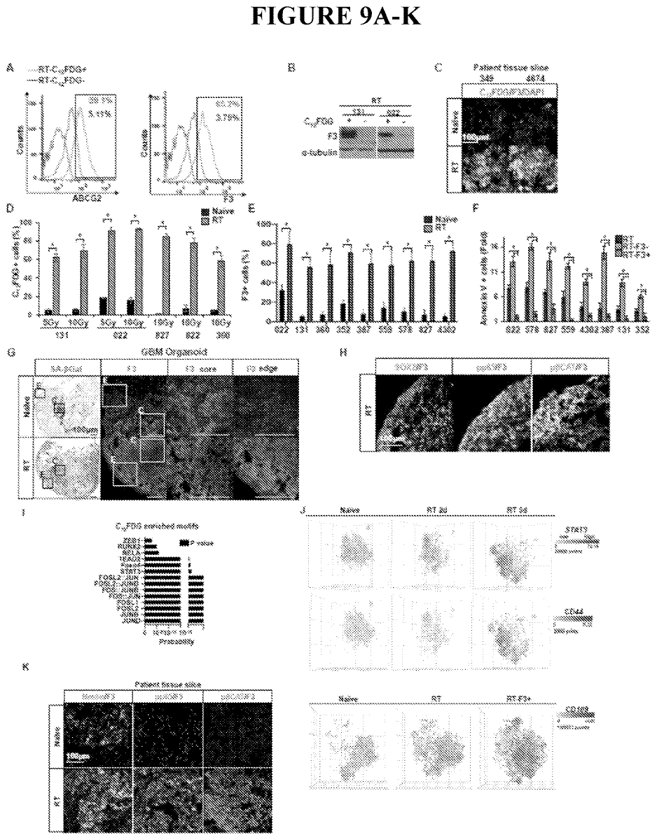

To investigate cellular states and molecular regulators of SA-βGal⁺ GBM cells, we profiled levels of cell surface proteins. Matched naïve and irradiated GBM cells (20 million cells each) were stained with C₁₂FDG, split into individual wells, and co-stained with each of 242 human clusters of differentiation (CD) antibodies (FIG. 2A). Co-staining results were quantitated by flow cytometry and expression level of each CD marker was calculated. Enriched cell surface receptors in RT-C12FDG⁺ cells included ABCG2, JAMA, PDGFR, CD109 and integrin proteins, implicated in stemness, GBM mesenchymal transition, and therapeutic resistance (FIG. 2B) (Bleau et al., 2009; Minata et al., 2019; Reya et al., 2001). The most enriched CD marker in our screen was CD142 (Tissue factor or F3), originally identified as a cell surface receptor that initiates blood coagulation (Morrissey et al., 1987). Tissue factor/F3 levels were increased over 10-fold in irradiated cells compared to the matched naïve cells, further increased in RT-C₁₂FDG⁺ cells (FIG. 2B). In the irradiated PDX tumor-bearing mice, we found the robust F3 induction, co-localized with C₁₂FDG⁺ tumor cells (FIG. 2C). This trend was further confirmed in GBM spheroids, GBM organoids and patient GBM slices (FIG. 9 A to G).

Two F3 canonical functions are initiation of blood coagulation and promotion of cell survival via intracellular signaling. Coagulation factor VII (F7, a cognate F3 ligand) is mainly produced in the liver and circulates in the blood stream as an inactive pro-enzyme. Upon interaction with F3, F7 is converted into a protease-active form (FVIIa), which then initiates coagulation by catalyzing a thrombin-producing protease cascade (Mackman, 2004; Rao and Rapaport, 1988). Intracellular signaling is mediated by activation of integrin and the protease-activated receptor (PAR), leading to activation of MAPK, PI3K, and NFκB (Cimmino and Cirillo, 2018; van den Berg et al., 2012). While F3 is expressed in various tumor types—including gliomas—and generally exerts pro-tumor effects (Bourcy et al., 2016; Unruh et al., 2019; Xie et al., 2022), the role(s) of F3 in RT responses and senescence are largely unknown.

To test whether post-irradiated F3^{high} cells are enriched for in vivo tumorigenic capacity, we isolated matched F3^{high} and F3^{low/-} cells from PDX tumors 5 days after in vivo radiation by F3 antibody-based cell sorting, and then injected these cells into immunodeficient mice. Compared to matched F3^{low/-} or mock-sorted bulk tumor cells, post-irradiated F3^{high} subpopulations yielded significantly higher capacities for tumor formation (FIGS. 2 D and E).

To gain insight into signaling nodes associated with F3, we performed ATAC (assay for transposase-accessible chromatin)-seq analysis to map genome regions with open chromatin structures in matched F3^{high} and F3^{low/-} subpopulations. The most enriched transcription factor-binding motifs in RT-F3⁺ GBM cells are STAT3 and FOSL2 (master transcriptional factors for the GBM mesenchymal (MES) transcriptional network), RELA (NFκB, a master regulator for senescence and GBM cell survival), as well as Foxo4 and TEAD2 (implicated in tumor dormancy and senescence) (Baar et al., 2017; Carro et al., 2010; Chien et al., 2011; Kurppa et al., 2020), most of which were also enriched in RT-C₁₂FDG⁺ GBM cells (FIGS. 2F and 9I). Following RT, GBM tumors undergo phenotypic transition toward MES subtype, which is mediated by activation of NFκB and STAT3 signaling (Bhat et al., 2013; Carro et al., 2010). We then determined the activation status of these pathways by immunoblots, immunostaining, and transcriptome analysis (FIGS. 2 G to L and 9 A to K). Consistent with ATAC data, irradiated F3^{high} GBM cells have elevated levels of active NFκB and STAT3 as well as stemness-associated proteins (Sox2, EZH2, and active β-catenin and integrin β1) (Kim et al., 2013; Prager et al., 2020) (FIGS. 2 G and H, and 9 H to K).

Active integrin signaling in this subpopulation is further supported by additional surface marker screening using anti-F3 antibody and 240 human CD markers. Similar to the C₁₂FDG co-staining data, several different integrin family proteins were highly enriched in irradiated F3^{high} GBM cells (Table S1). In addition, RT-F3⁺ cells have high levels of GBM mesenchymal markers (YKL-40, CD44, and active STAT3) (FIGS. 2J, 2K and 9J) (Aldape et al., 2019). Lastly, secretome profiling and gene signature analysis showed that irradiated F3^{high} GBM cells secreted significantly higher levels of SASP factors such as IL6, IL8, HGF, and EGF, relative to bulk naïve or irradiated tumor cells (FIGS. 2 L and M). Together, these data indicate that the RT-F3⁺ populations have enriched traits of sternness, mesenchymal GBM cell transition, senescence-like epigenomic reprogramming, and SASP.

### F3 Signaling Primes Radiation-Induced Changes Both in GBM and the TME.

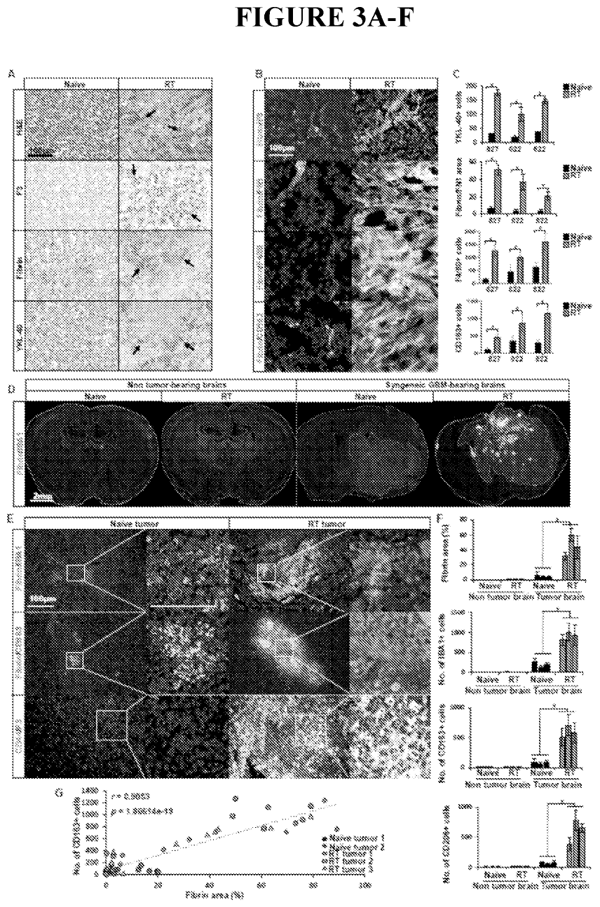

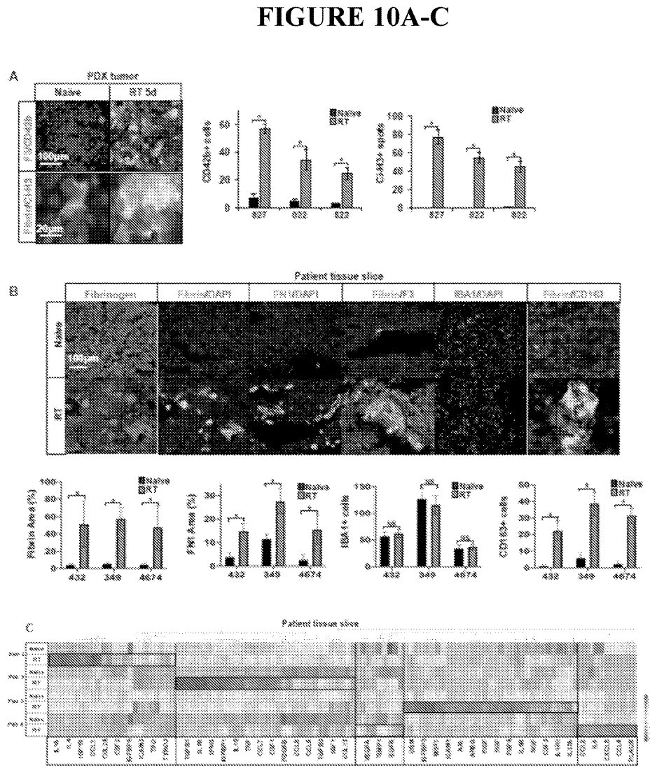

To probe the roles of F3 in GBM radiation responses in vivo, we performed immunohistochemical staining analyses using human PDX, GBM slice, and syngeneic mouse glioma models (FIG. 3). Orthotopic PDX tumor-bearing mice were irradiated, and the brain tissues harvested 5 days later. In regions with a robust upregulation of F3, we found that F3 positively correlated with high levels of fibrin complexes, a key effector in the P3-initiated coagulation cascade (FIG. 3A). F3-positive GBM cells were also positive for YKL-40 and CD44 (MES-like GBM markers) and fibronectin 1 (FN1, an ECM protein and MES-like GBM marker) (Bhat et al., 2013; Phillips et al., 2006; Wang et al., 2017). Fibrin polymer and ECM molecules such as FN1 are known to form a mesh-like structure together, referred to as provisional ECM, which provides a scaffold for recruitment of macrophages, activated platelets, and neutrophil extracellular traps (NETs) (Galmiche et al., 2022). Indeed, we found significant increases in the numbers of TAMs and M2-like TAMs (CD163⁺) in F3-positive, fibrin/FN1 complex-rich regions in the irradiated tumors (FIGS. 3B, 3C, and 10A).

Human GBM slices resected from newly diagnosed GBM patients maintain in vivo tumor architecture and TME including immune cells, endothelial cells, and astrocytes; thus they can mimic acute responses of human GBM in situ, albeit for a few days (LeBlanc et al., 2022). We prepared acute GBM slices within 6 hours post-surgery, irradiated them ex vivo, and then processed for immunostaining and cytokine array analysis 3 days later (FIG. 10B). Fibrinogen (precursor of fibrin) and F7 were detected in these GBM slices. Irradiation resulted in robust induction of fibrin/FN1 complexes and CD163⁺ macrophages. Cytokine analysis utilizing 4 sets of matched naïve and irradiated GBM tissue slices consistently showed higher levels of secreted proteins in the conditioned media from irradiated GBM slices (FIG. 10C). These data suggest a causal relationship between that RT and fibrin polymerization, an enhanced secretory phenotype, and TAM polarization.

To validate the above findings in the intact immune microenvironment, we employed a PDGFβ-driven, p53-null, syngeneic mouse glioma, a representative proneural subtype tumor (Hambardzumyan et al., 2009). Naïve tumors showed relatively low basal levels of fibrin, TAMs and CD44⁺ cells. Upon irradiation, however, we found massive increases in fibrin polymerization and TAM infiltration, as well as in levels of F3 and CD44 (FIG. 3 D to F). Notably, most of the CD163⁺, CD206⁺ M2-like TAMs were detected in the fibrin polymer-enriched, F3⁺, CD44⁺ tumor regions, indicating a strong positive correlation between them (FIG. 3G). Together, these data further suggest a link between RT-induced coagulation, oncogenic TAMs, and GBM mesenchymal transition.

### Molecular Mechanisms of F3 Signaling in GBM Reprogramming and Radio-Resistance

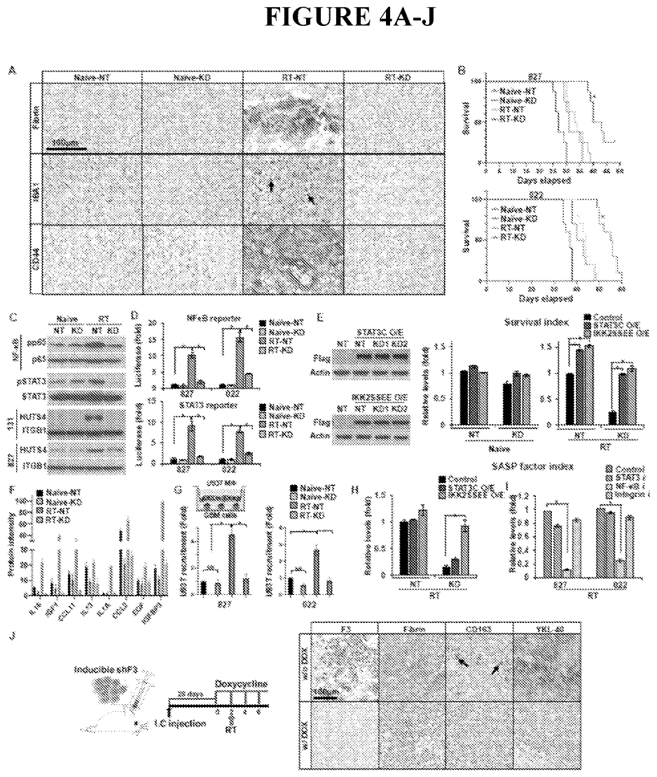

Given strong spatiotemporal associations between F3 and RT-induced GBM remodeling (FIG. 3), we first determined the roles of F3 in vivo via shRNA-mediated F3 knockdown. Non-targeting shRNA or F3 knockdown (KD) shRNA-expressing GBM cells were transplanted into the brains of nude mice and in vivo radiation was started 20 days later (FIGS. 4 A and B). Immunostaining analysis of the tumor-bearing brain sections revealed that F3 suppression significantly abrogated RT-induced fibrin polymers and accumulation of IBA1⁺ and CD44⁺ cells (FIG. 4A). F3 knockdown or irradiation alone extended the survival of tumor-bearing mice compared to the control group. Notably, the group injected with F3 KD cells and given radiation showed far longer survival than all other groups (p<0.001 by log-rank analysis) (FIG. 4B).

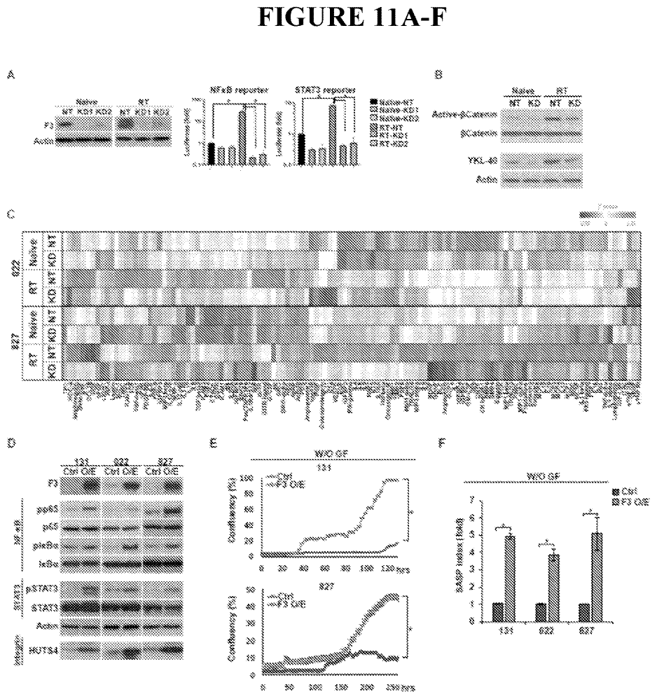

We then determined the effects of F3 knockdown on the activation status of NFκB, STAT3, and integrin signaling (FIGS. 4 and 11). In the naïve state, F3 knockdown did not induce significant decreases in pp65 and pSTAT3, possibly reflecting low levels of F3. In contrast, F3 knockdown potently impeded RT-induced upregulation of NFκB, STAT3, and integrin activities, as well as mesenchymal traits (YKL-40 and FN1) (FIGS. 4 C and D, 11 A and B). Conversely, over-expression of constitutively active mutants of NFκB or STAT3 signaling rescued cell survival of RT-F3 KD cells, indicating that both NFκB and STAT3 activities are key downstream effectors of RT-induced F3 signaling (FIG. 4E).

As RT-F3⁺ GBM cells secrete high levels of SASP factors (FIG. 2M) and NFκB activity is a major regulator of SASP factor secretion, we determined the levels of cytokines/chemokines secreted by F3 KD GBM cells with or without irradiation. RT-induced upregulation of multiple cytokine/chemokines was significantly reduced by F3 knockdown (FIGS. 4F and 11C). To investigate the roles of F3 in chemokine-mediated TAM recruitment, we adapted in vitro transwell assays using U937 macrophage-like cells (primed U937) (Koren et al., 1979). Conditioned media from irradiated GBM cells attracted significantly larger numbers of U937 cells compared to conditioned media from matched naïve cells. F3 knockdown reduced the levels of macrophage recruitment by ~80% (FIG. 4G). Forced activation of NFκB activity but not STAT3 rescued SASP factor secretion in irradiated F3 knockdown cells and NFκB suppression by p65 shRNA significantly inhibited RT-induced SASP factor secretion, suggesting that RT-induced SASP in GBM is largely dependent on NFκB signaling (FIGS. 4 H and I).

Conversely, over-expression of F3 in naïve GBM cells showed elevated levels of active NFκB, STAT3 and integrin signaling (FIG. 11D). Consistent with this, F3 over-expressing cells were more proliferative in the culture media without the added growth factors and these cells had the enhanced SASP factor secretion compared to the control (FIGS. 11 E and F). Lastly, the roles of F3 in RT responses in tumor and TME in vivo were further confirmed in a doxycycline-mediated inducible F3 KD system (FIG. 4J). These data support that F3 is a critical regulator for the survival and cell state transition of irradiated GBM cells, as well as for SASP factor secretion.

### Recombinant ΔFVII Protein Impeded Radiation-Induced Coagulation, SASP Factor Secretion, and TAM Accumulation In Vivo.

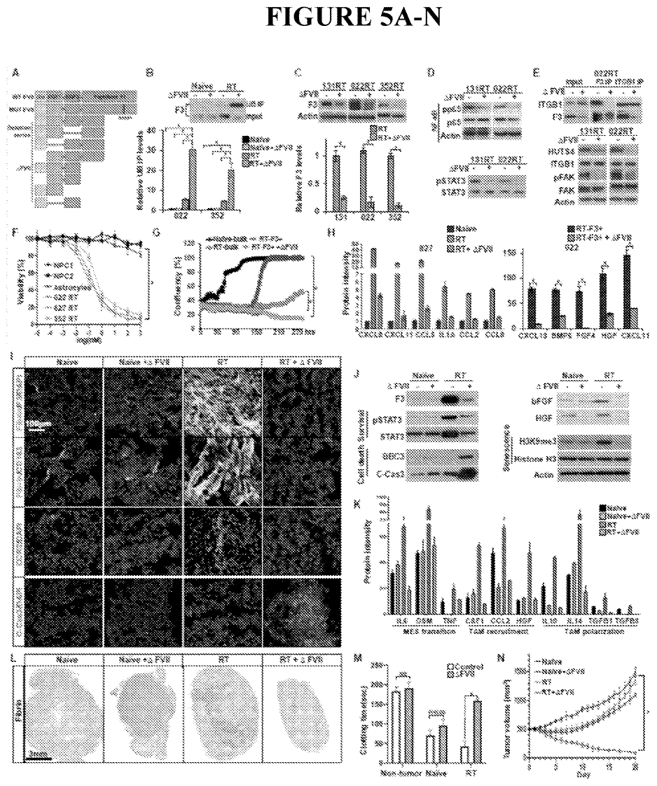

The above data collectively suggest that F3 signaling is a critical regulator for post-RT tumor growth and drives an oncogenic TME, making it a therapeutic target. Since F3 protein, after binding to its ligand F7, is eventually degraded via the ubiquitin pathway, we hypothesized that specific F7 derivatives may directly trigger F3 degradation without eliciting F3-mediated oncogenic effects (Ettelaie et al., 2016). To test, we over-expressed a series of F7 deletion mutants in GBM cells via lentiviral transduction and determined the proliferation of these GBM cells after irradiation (FIG. 5A). Through this screen, we found a deletion mutant that potently impeded the growth of irradiated GBM cells, which we designated as ΔFVII (188-amino acid protein without the F7 protease domain, but counting the 20 amino acid signal peptide).

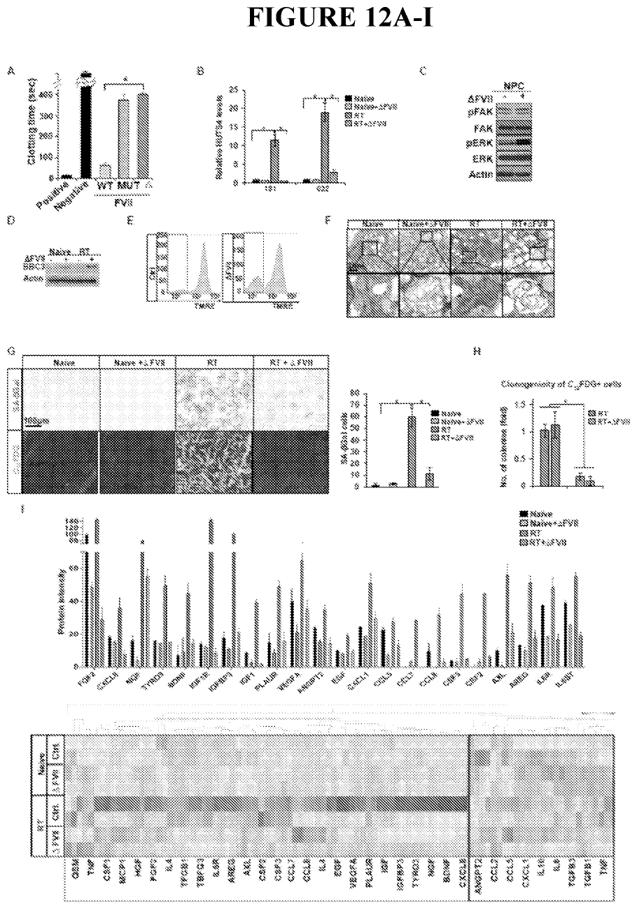

ΔFVII recombinant protein lacked the pro-coagulation activity unlike wild-type F7 or FVIIa, as determined by in vitro coagulation assays (FIG. 12A). Instead, ΔFVII robustly induced ubiquitin-mediated degradation of F3 proteins and significantly reduced the level of F3 proteins in irradiated GBM cells (FIGS. 5 B and C). Consistent with this, irradiated GBM cells treated with ΔFVII for a day showed a significant decrease in phosphorylated p65 and STAT3 proteins (FIG. 5D). Furthermore, we found that ΔFVII decreased the levels of the co-immunoprecipitated F3-integrin complexes, HUTS4 (specific for active integrin β1), and phosphorylated FAK (an immediate downstream effector of integrin signaling) (FIGS. 5E and 12B). We then determined the effects of ΔFVII on survival and clonogenic growth of GBM cells (FIGS. 5 F and G). ΔFVII treatment effectively impaired survival of irradiated GBM cells with an IC₅₀ in the 0.1 to 1 nM range (FIG. 5F). In contrast, normal neural progenitor cells (NPCs) and primary astrocytes did not exhibit cytotoxicity even at micromolar ΔFVII concentration (FIG. 5F). Unlike GBM cells, normal brain cells showed little change in the levels of pFAK and pERK after ΔFVII treatment, partially explaining GBM-specific cytotoxicity of ΔFVII (FIG. S12C). Real-time cell imaging analysis demonstrated that ΔFVII treatment potently impaired clonogenic growth of irradiated F3^{high} GBM cells (FIG. 5G). GBM cells treated with ΔFVII showed disruption of mitochondrial membrane potential and defective mitochondrial structure (FIG. 12 D to F). Lastly, to determine the effects of ΔFVII on SASP factor secretion, we treated irradiated GBM cells with ΔFVII for 1 day and collected the conditioned culture media. ΔFVII treatment significantly diminished the secretion of cytokines (FIG. 5H). Together, these data suggest that ΔFVII is a potent anti-GBM agent that mitigates F3-mediated oncogenic signaling, especially in combination with RT.

To determine in vivo effects of ΔFVII, we started with subcutaneous tumor models. We irradiated human PDX tumors focally when the tumors reached ~500 mm³ and administered ΔFVII protein (50 μg/kg body weight) via intravenous injection. Compared to the naïve controls, tumors harvested 5 days after radiation showed marked increases in fibrin/FN1 complexes, tumor-infiltrating CD11b⁺, F4/80⁺, CCR2⁺ immune cells and CD163⁺ M2-like TAMs, along with massive upregulation of F3 (FIG. 5I and data not shown). Notably, ΔFVII treatment potently inhibited all the above changes, with a dramatic increase in the number of cleaved-caspase 3 (C-Cas3)⁺ cells (FIG. 5I). To evaluate in vivo effects of ΔFVII on signaling pathway activation and senescence-like characteristics, we harvested the tumors from each group and processed for further analyses. ΔFVII treatment significantly reduced RT-induced cell survival signaling such as STAT3, but increased levels of cell death-associated proteins in tumor (FIG. 5J). RT-induced SA-βGal⁺ reactivities were significantly decreased by ΔFVII treatment and clonogenic capacities of sorted RT-C₁₂FDG⁺ cells isolated from these tumors were significantly abolished by in vivo ΔFVII treatment (FIGS. 12 G and H). To analyze the chemokine/cytokine microenvironment within the tumors treated with radiation, ΔFVII, or both agents, we performed cytokine profiling assays using tumor lysates. Irradiation induced significant upregulation of secreted proteins, many of which have well-known functions for macrophage recruitment, M2-like TAM polarization, and cell state transition. Notably, ΔFVII treatment significantly and globally repressed the chemokine/cytokine levels (FIGS. 5K and 12I).

To determine the effects of tumors and ΔFVII on systemic hemostasis, we measured the blood clotting activities by standard tail bleeding assays (FIG. 5M). Compared to the control mice, tumor-bearing mice had shorter blood clotting time, which was further shortened by irradiation. While ΔFVII treatment on non-tumor bearing mice did not affect blood clotting time, the combination of irradiation and ΔFVII reverted hypercoagulation state in the RT-tumor bearing mice to near-normal range (FIG. 5M). Studies of tumor growth kinetics showed that irradiation or ΔFVII monotherapy alone suppressed tumor growth rate to about 50 to 60% of that of naïve tumors, but the effect of either agent alone was transient and led to rapid regrowth (FIGS. 5L and 5N). In contrast, combination therapy yielded near-complete tumor regression (FIG. 5N).

### ΔFVII Therapies Radio-Sensitize GBM Tumors in Orthotopic PDX Models.

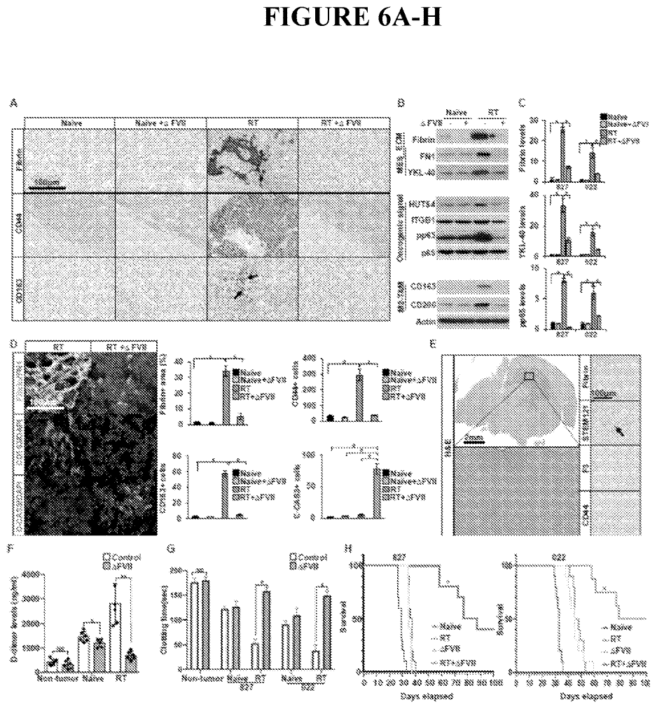

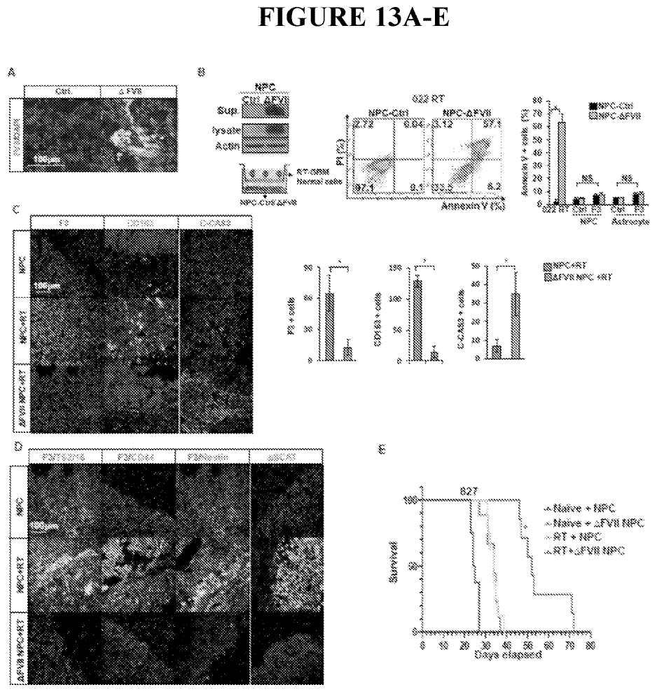

We tested the effects of ΔFVII recombinant protein in orthotopic GBM PDX models using patient-derived 022 or 827 GBM cells. Irradiation was started 20 days (for 827 tumor) or 25 days (for 022 tumor) after tumor cell implantation and ΔFVII protein was delivered via intravenous injection, concurrently with irradiation (FIGS. 6A and 13A). Five days after irradiation, the brains from each group were harvested for immunostaining (n=3) and immunoblot analysis through tumor dissection (n=3). Combination treatment with irradiation and ΔFVII greatly diminished RT-induced fibrin accumulation, MES cell state transition, TAM polarization, and activities of integrin and NFκB signaling, but increased numbers of C-cas3⁺ cells (FIG. 6 A to D). Notably, histological examination of the brain sections from the mice receiving combined treatment at 38 days after implantation (the time when RT-treated mice were sacrificed) showed few tumor cells and only faint staining for fibrin polymers (FIG. 6E). We also determined the effects of orthotopic GBM tumors and ΔFVII on systemic hemostasis by D-dimer and tail bleeding assays (FIGS. 6 F and G). Similar to subcutaneous tumor results (FIG. 5M), ΔFVII treatment potently inhibited the hypercoagulatory state in RT-tumor bearing mice (FIGS. 6 F and G). Lastly, irradiation or ΔFVII monotherapy alone extended survival of tumor-bearing mice by about 10 days compared to the control group (FIG. 6H). Notably, the group that received combination therapy showed a far greater survival extension, with about 50% of mice surviving and lacking detectable tumors three months later (FIG. 6H).

While several studies have reported that the blood-brain barrier (BBB) is functionally disrupted in some GBM (Arvanitis et al., 2020), the highly infiltrative GBM cells in the neighboring brain parenchyma presents technical challenges in developing effective anti-GBM therapeutics. As an independent but complementary therapeutic approach to ΔFVII recombinant protein, we developed a neural progenitor cell (NPC)-based cellular vector system. NPCs have intrinsic tumor-homing properties and survive well in the brains (Bago et al., 2017; Kauer et al., 2011). Lentivirally transduced ΔFVII-expressing NPCs were viable and maintained stable levels of secreted ΔFVII (FIG. 13B). Upon co-culture with irradiated GBM cells, ΔFVII-expressing NPCs but not the control NPCs induced massive GBM cell death (FIG. 13B). To mimic a clinical scenario in newly diagnosed human glioma patients, we injected ΔFVII-expressing NPCs into the established PDX tumors, followed by head-only irradiation two days later (FIGS. 13 C and D). Similar to the RT and ΔFVII combination, co-treatment with RT and ΔFVII-NPCs greatly diminished RT-induced F3 induction and activities of pro-tumorigenic signaling, leading to longer survival of tumor-bearing mice (FIGS. 13 C to E).

Recurrent Tumors from Early-Relapse GBM Patients Harbor the Upregulated Gene Signatures for the Senescence and Coagulation Pathways.

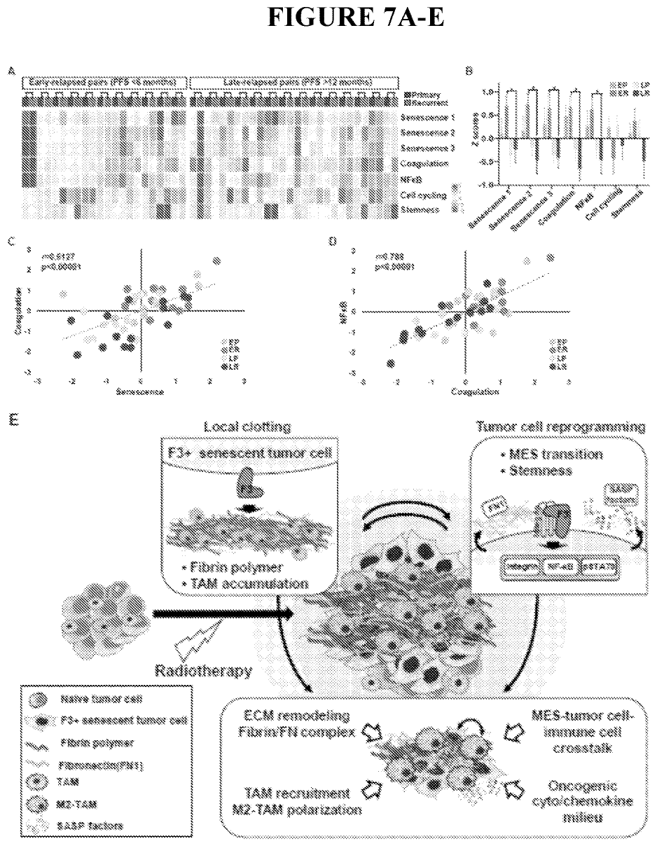

Comparison of matching primary and recurrent GBMs can inform therapy-induced phenotypic tumor evolution, including GBM cell state and associations between TME components. We therefore analyzed transcriptional profiles of primary GBMs (n=25) treated with radiation therapy and separated early relapses (Progression-free survival (PFS)<6 months, n=11) from late relapses (PFS>12 months, n=14) using the dataset from GLASS consortium (FIG. 7) (Varn et al., 2022; Wang et al., 2017). The status of pathway activation in each tumor was inferred by the pathway enrichment scores from representative gene signature sets (Bhat et al., 2013; Coppe et al., 2008; Fridman and Tainsky, 2008; Magnus et al., 2013) (FIGS. 7 A and B). We observed no significant differences in signature gene set levels between primary tumors with early and late relapse. When comparing relapsed GBMs, however, we found that early-relapse GBMs showed a significantly higher predicted presence of M2 macrophages and the enrichment of senescence, coagulation, and NFκB signatures compared with late-relapse GBMs (Wang et al., 2017) (FIG. 7B). Furthermore, the enrichment scores for senescence, coagulation, and NFκB signatures highly correlated with each other, especially in the early-relapse tumor pairs (FIGS. 7 C and D). These data are consistent with our findings and may provide clinical relevance for potential translation of ΔFVII-based therapies.

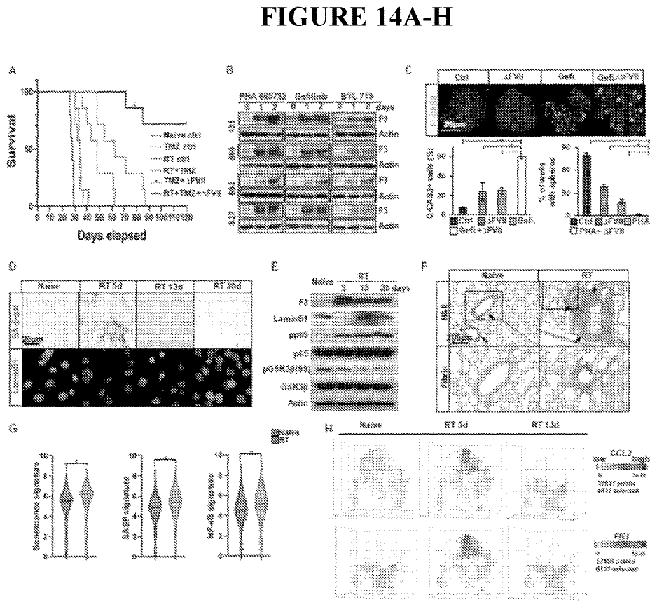

We then determined whether F3 targeting can be combined with other anti-cancer therapeutic approaches. A combination therapy with TMZ and ΔFVII, and triple combination of RT, TMZ, and ΔFVII significantly prolonged the survival of tumor-bearing mice (FIG. 14A). Aberrant activation of the EGFR, MET, and AKT pathways is frequently found in GBM, and each pathway has been established as a potential therapeutic target (Brennan et al., 2013; Liau et al., 2017). We treated 4 different GBM cells with representative EGFR, MET, and AKT inhibitors and ΔFVII (FIG. 14B). Combination treatment robustly induced tumor cell death and impaired the clonogenic growth of GBM cells to a much greater degree than monotherapy alone (FIG. 14C). Lastly, pre-metastatic MG63 osteosarcoma model revealed RT-induced F3 signaling very similar to GBMs, raising the possibility that our findings on RT-induced F3 signaling may be applicable to other cancer types (FIG. 14 D to H).

Our data collectively support the previously unidentified concept that activation of F3 signaling during therapy-induced senescence is a central initiator to trigger global adaptation programs in tumor cells and in the TME, leading to therapy resistance and tumor recurrence (FIG. 7E).

We show that radiation-induced SA-βGal+ GBM cells robustly contribute to post-RT tumor regrowth by active clonal expansion and global reorganization of immune, ECM, and cytokine landscapes in the TME. F3 proteins are rapidly elevated in the SA-βGal⁺ GBM cells upon irradiation. F3 signaling promotes clonal expansion, mesenchymal-like cellular state transition, and secretion of oncogenic SASP factors and ECM proteins. Concurrently, F3 also initiates a hyperactive coagulation cascade including local fibrin polymers, which in turn facilitates TAM accumulation/polarization and ECM remodeling in the tumor regions. These F3-initiated cellular events are functionally linked, and together they constitute an oncogenic feed-forward loop, in which TAMs and SASP factors are critical players. We also demonstrate a strategy to inhibit oncogenic F3 signaling as an anti-GBM therapeutic approach. These data collectively support that F3 is a critical driver of oncogenic senescence and therapeutic resistance in GBM, opening potential therapeutic avenues for F3 targeting.

### Tumor Cell Senescence

At the early stages of tumor initiation, senescence is an essential tumor-suppressive barrier that limits expansion of pre-malignant cells. It has been generally thought that as tumors evolve to malignant forms, the proportion of senescent cells is low (Collado et al., 2005). It is increasingly clear now that various therapies including irradiation, chemotherapeutics, and targeted inhibitors induce massive numbers of senescent-like cells in tumors (Ewald et al., 2010; Prasanna et al., 2021), however, senescent-like tumor cells have been identified by colorimetric SA-βGal assay and very limited senescent cell marker staining in most studies.

We report here the existence of an F3-driven phenotype in GBM sharing some features of senescent cells—including SA-βGal reactivity, senescence marker expression (HP1γ and H3K9me3), enrichment of representative senescence gene sets, and SASP-like secretory phenotype—with features of GBM stem cells. The enhanced DNA damage responses and clonal expansion of irradiated SA-βGal⁺ GBM cells suggest that GBM cell senescence is not consistent with irreversible cell cycle arrest, a central tenet of cellular senescence. Recent studies showing that the senescence program activates key stemness signaling pathways such as WNT and YAP (Kurppa et al., 2020; Milanovic et al., 2018) and the senescent state in cancer is highly dynamic and reversible (Kurppa et al., 2020) are highly consistent with our findings. GBM harbor defects in apoptosis and cell cycle regulators such as homozygous deletion of Cdkn2a and deregulated TP53 pathways. Thus, the senescence-like tumor cell phenotype without stable cell cycle arrest can be a trait of TIS in other malignant cancers.

Increased blood clotting activities and aberrant fibrin clots have frequently been observed in patients with senescence-associated pathologies including COVID, tissue injury, and aging (Lee et al., 2021; Moiseeva et al., 2023; Nguyen et al., 2022; Wiley et al., 2019). Our findings to indicate the roles of F3 in RT-SA-βGal+ GBM cells may suggest the possibility that senescence plays a causal role in hypercoagulation activity in non-cancer pathologies.

### Roles of F3 in Cancer

The close relationship between cancer and thrombosis has been recognized by Trousseau since 1865 (Trousseau, 1865). Hyper-coagulability can lead to serious, life-threatening conditions such as venous thromboembolism (VTE). VTE is frequently found in cancer patients, among whom high-grade glioma patients showed the highest incidence (Galmiche et al., 2022; Saidak et al., 2021). F3, originally identified as an initiator of a stress-responsive coagulation cascade, has been implicated in hyper-coagulation activities in cancer, pro-proliferative signaling, and tumor dormancy, and metastasis (Bourcy et al., 2016; Cimmino and Cirillo, 2018; Feinauer et al., 2021; Galmiche et al., 2022; Morrow et al., 2018). A more recent study reported that F3 is preferentially expressed in quiescent stem-like GBM cells (Xie et al., 2022). Molecular details of F3 signaling, however, are incompletely understood. Our findings may provide a step forward to a better understanding of F3 signaling, which may be critical for advancing therapeutic strategies.

Anti-Cancer Therapeutic Approaches that Target Senescence-Associated Pathways

Various therapeutic approaches that target senescent tumor cells and/or senescence-associated pathways have been developed as potential anticancer approaches (Prasanna et al., 2021). These approaches can be largely classified into three categories: (i) targeting individual oncogenic SASP factors (Birch and Gil, 2020; Dou et al., 2017), (ii) targeting individual stemness/survival pathways that are particularly induced by the TIS program (Kurppa et al., 2020; Wang et al., 2019), and (iii) targeting senescent tumor cells with senolytic approaches (Amor et al., 2020).

Similar to the highly overlapping RTK signaling networks found in cancer, post-therapy malignant tumors secrete multiple SASP factors with very similar or redundant functions (Birch and Gil, 2020; Coppe et al., 2010; De Palma and Lewis, 2013; DeNardo and Ruffell, 2019; Faget et al., 2019; Hara et al., 2021; Wang et al., 2020). TIS-associated epigenetic reprogramming may reactivate multiple, potentially redundant, oncogenic transcription networks (Kurppa et al., 2020; Milanovic et al., 2018; Wang et al., 2019). Our data suggest that ΔFVII targeting approach is a viable therapeutic option. F3 targeting potently inhibited radiation-induced TAM accumulation and oncogenic TAM polarization, indicating a potential intersection with TAM re-education approaches and/or other immune-based anti-cancer approaches. We postulate that F3 signaling is a crucial initiator to trigger multiple oncogenic pathways in therapy-induced tumor cell senescence setting.

As senescence in the brain parenchyma and immune landscapes are likely critical for RT-induced remodeling and GBM radio-resistance, extensive studies in syngeneic tumor models can provide deeper understanding of associations between immune, microenvironmental components and treatment response. Most of our data were obtained from PDX-bearing immunocompromised mice models. Despite this caveat, our data reveal that F3 signaling in GBM is an active adaptation program to evade therapeutic pressure and potentiate oncogenic effects of therapy-induced senescence. Thus, inhibiting F3 signaling is a promising strategy to markedly enhance otherwise suboptimal anti-GBM therapies.

### REFERENCES

Ahmed, et al., (2013). Harnessing the potential of radiation-induced immune modulation for cancer therapy. Cancer Immunol Res 1, 280-284.
Aldape, et al. (2019). Challenges to curing primary brain tumours. Nat Rev Clin Oncol 16, 509-520.
Amor et al. (2020). Senolytic CAR T cells reverse senescence-associated pathologies. Nature 583, 127-132.
Arvanitis, et al., (2020). The blood-brain barrier and blood-tumour barrier in brain tumours and metastases. Nat Rev Cancer 20, 26-41.
Baar et al. (2017). Targeted Apoptosis of Senescent Cells Restores Tissue Homeostasis in Response to Chemotoxicity and Aging. Cell 169, 132-147 e116.
Bago et al., (2017). Tumor-homing cytotoxic human induced neural stem cells for cancer therapy. Sci Transl Med 9.
Bao et al., (2006). Glioma stem cells promote radioresistance by preferential activation of the DNA damage response. Nature 444, 756-760.
Barker et al., (2015). The tumour microenvironment after radiotherapy: mechanisms of resistance and recurrence. Nat Rev Cancer 15, 409-425.
Ben-Porath et al., (2008). An embryonic stem cell-like gene expression signature in poorly differentiated aggressive human tumors. Nat Genet 40, 499-507.
Bhang et al. (2015). Studying clonal dynamics in response to cancer therapy using high-complexity barcoding. Nat Med 21, 440-448.
Bhat et al. (2013). Mesenchymal differentiation mediated by NF-kappaB promotes radiation resistance in glioblastoma. Cancer Cell 24, 331-346.
Birch, J., and Gil, J. (2020). Senescence and the SASP: many therapeutic avenues. Genes Dev 34, 1565-1576.
Bleau et al., (2009). PTEN/PI3K/Akt pathway regulates the side population phenotype and ABCG2 activity in glioma tumor stem-like cells. Cell Stem Cell 4, 226-235.
Bourcy et al. (2016). Tissue Factor Induced by Epithelial-Mesenchymal Transition Triggers a Procoagulant State That Drives Metastasis of Circulating Tumor Cells. Cancer Res 76, 4270-4282.
Brennan et al. (2013). The somatic genomic landscape of glioblastoma. Cell 155, 462-477.
Carro et al. (2010). The transcriptional network for mesenchymal transformation of brain tumours. Nature 463, 318-325.
Chen et al., (2012). Malignant glioma: lessons from genomics, mouse models, and stem cells. Cell 149, 36-47.
Chien et al. (2011). Control of the senescence-associated secretory phenotype by NF-kappaB promotes senescence and enhances chemosensitivity. Genes Dev 25, 2125-2136.
Cimmino, G., and Cirillo, P. (2018). Tissue factor: newer concepts in thrombosis and its role beyond thrombosis and hemostasis. Cardiovasc Diagn Ther 8, 581-593.
Collado et al. (2005). Tumour biology: senescence in premalignant tumours. Nature 436, 642.
Coppe et al., (2010). The senescence-associated secretory phenotype: the dark side of tumor suppression. Annu Rev Pathol 5, 99-118.
Coppe et al., (2008). Senescence-associated secretory phenotypes reveal cell-nonautonomous functions of oncogenic RAS and the p53 tumor suppressor. PLoS Biol 6, 2853-2868.
De Palma, M., and Lewis, C. E. (2013). Macrophage regulation of tumor responses to anticancer therapies. Cancer Cell 23, 277-286.
Debacq-Chainiaux, et al., (2009). Protocols to detect senescence-associated beta-galactosidase (SA-betagal) activity, a biomarker of senescent cells in culture and in vivo. Nat Protoc 4, 1798-1806.
DeNardo, D. G., and Ruffell, B. (2019). Macrophages as regulators of tumour immunity and immunotherapy. Nat Rev Immunol 19, 369-382.
Dimri et al. (1995). A biomarker that identifies senescent human cells in culture and in aging skin in vivo. Proc Natl Acad Sci USA 92, 9363-9367.
Ding et al. (2013). Cancer-Specific requirement for BUB1B/BUBR1 in human brain tumor isolates and genetically transformed cells. Cancer Discov 3, 198-211.
Dou et al. (2017). Cytoplasmic chromatin triggers inflammation in senescence and cancer. Nature 550, 402-406.
Ettelaie et al., (2016). Oligoubiquitination of tissue factor on Lys255 promotes Ser253-dephosphorylation and terminates TF release. Biochim Biophys Acta 1863, 2846-2857.
Ewald et al., (2010). Therapy-induced senescence in cancer. J Natl Cancer Inst 102, 1536-1546.
Faget, D. V., Ren, Q., and Stewart, S. A. (2019). Unmasking senescence: context-dependent effects of SASP in cancer. Nat Rev Cancer 19, 439-453.
Feinauer et al. (2021). Local blood coagulation drives cancer cell arrest and brain metastasis in a mouse model. Blood 137, 1219-1232.
Fridman, A. L., and Tainsky, M. A. (2008). Critical pathways in cellular senescence and immortalization revealed by gene expression profiling. Oncogene 27, 5975-5987.
Galmiche et al., (2022). Coagulome and the tumor microenvironment: an actionable interplay. Trends Cancer.
Gilbert et al. (2014). A randomized trial of bevacizumab for newly diagnosed glioblastoma. N Engl J Med 370, 699-708.
Gorgoulis et al. (2019). Cellular Senescence: Defining a Path Forward. Cell 179, 813-827.
Greaves, M., and Maley, C. C. (2012). Clonal evolution in cancer. Nature 481, 306-313.
Halliday et al., (2014). In vivo radiation response of proneural glioma characterized by protective p53 transcriptional program and proneural-mesenchymal shift. Proc Natl Acad Sci USA 111, 5248-5253.
Hambardzumyan et al., (2009). Modeling Adult Gliomas Using RCAS/t-va Technology. Transl Oncol 2, 89-95.
Hara et al. (2021). Interactions between cancer cells and immune cells drive transitions to mesenchymal-like states in glioblastoma. Cancer Cell 39, 779-792 e711.
He, S., and Sharpless, N. E. (2017). Senescence in Health and Disease. Cell 169, 1000-1011.
Hubert et al., (2016). A Three-Dimensional Organoid Culture System Derived from Human Glioblastomas Recapitulates the Hypoxic Gradients and Cancer Stem Cell Heterogeneity of Tumors Found In Vivo. Cancer Res 76, 2465-2477.
Jacob et al. (2020). A Patient-Derived Glioblastoma Organoid Model and Biobank Recapitulates Inter- and Intra-tumoral Heterogeneity. Cell 180, 188-204 e122.
Kauer et al., (2011). Encapsulated therapeutic stem cells implanted in the tumor resection cavity induce cell death in gliomas. Nat Neurosci 15, 197-204.
Kennedy, A. R., Maity, A., and Sanzari, J. K. (2016). A Review of Radiation-Induced Coagulopathy and New Findings to Support Potential Prevention Strategies and Treatments. Radiat Res 186, 121-140.
Kim et al. (2013). Phosphorylation of EZH2 activates STAT3 signaling via STAT3 methylation and promotes tumorigenicity of glioblastoma stem-like cells. Cancer Cell 23, 839-852.
Koren, H. S., Anderson, S. J., and Larrick, J. W. (1979). In vitro activation of a human macrophage-like cell line. Nature 279, 328-331.
Kurppa et al. (2020). Treatment-Induced Tumor Dormancy through YAP-Mediated Transcriptional Reprogramming of the Apoptotic Pathway. Cancer Cell 37, 104-122 e112.
LeBlanc et al. (2022). Single-cell landscapes of primary glioblastomas and matched explants and cell lines show variable retention of inter- and intratumor heterogeneity. Cancer Cell 40, 379-392 e379.
Lee et al. (2021). Virus-induced senescence is a driver and therapeutic target in COVID-19. Nature 599, 283-289.
Liau et al. (2017). Adaptive Chromatin Remodeling Drives Glioblastoma Stem Cell Plasticity and Drug Tolerance. Cell Stem Cell 20, 233-246 e237.
Lim, M., Xia, Y., Bettegowda, C., and Weller, M. (2018). Current state of immunotherapy for glioblastoma. Nat Rev Clin Oncol 15, 422-442.
Mackman, N. (2004). Role of tissue factor in hemostasis, thrombosis, and vascular development. Arterioscler Thromb Vasc Biol 24, 1015-1022.
Magnus, N., Gerges, N., Jabado, N., and Rak, J. (2013). Coagulation-related gene expression profile in glioblastoma is defined by molecular disease subtype. J Thromb Haemost 11, 1197-1200.
Milanovic et al. (2018). Senescence-associated reprogramming promotes cancer stemness. Nature 553, 96-100.
Minata et al. (2019). Phenotypic Plasticity of Invasive Edge Glioma Stem-like Cells in Response to Ionizing Radiation. Cell Rep 26, 1893-1905 e1897.
Moiseeva et al. (2023). Senescence atlas reveals an aged-like inflamed niche that blunts muscle regeneration. Nature 613, 169-178.
Morrissey, J. H., Fakhrai, H., and Edgington, T. S. (1987). Molecular cloning of the cDNA for tissue factor, the cellular receptor for the initiation of the coagulation protease cascade. Cell 50, 129-135.
Morrow et al. (2018). Positively selected enhancer elements endow osteosarcoma cells with metastatic competence. Nat Med 24, 176-185.
Neftel et al. (2019). An Integrative Model of Cellular States, Plasticity, and Genetics for Glioblastoma. Cell 178, 835-849 e821.
Nguyen, D., Jeon, H. M., and Lee, J. (2022). Tissue factor links inflammation, thrombosis, and senescence in COVID-19. Sci Rep 12, 19842.
Phillips et al. (2006). Molecular subclasses of high-grade glioma predict prognosis, delineate a pattern of disease progression, and resemble stages in neurogenesis. Cancer Cell 9, 157-173.
Prager et al., (2020). Glioblastoma Stem Cells: Driving Resilience through Chaos. Trends Cancer 6, 223-235.
Prasanna et al. (2021). Therapy-Induced Senescence: Opportunities to Improve Anticancer Therapy. J Natl Cancer Inst 113, 1285-1298.
Rao, L. V., and Rapaport, S. T. (1988). Activation of factor VII bound to tissue factor: a key early step in the tissue factor pathway of blood coagulation. Proc Natl Acad Sci USA 85, 6687-6691.
Reya, T., Morrison, S. J., Clarke, M. F., and Weissman, I. L. (2001). Stem cells, cancer, and cancer stem cells. Nature 414, 105-111.
Saidak et al., (2021). A pan-cancer analysis of the human tumor coagulome and its link to the tumor immune microenvironment. Cancer Immunol Immunother 70, 923-933.
Schmitt, C. A., Wang, B., and Demaria, M. (2022). Senescence and cancer—role and therapeutic opportunities. Nat Rev Clin Oncol 19, 619-636.
Stupp et al. (2005). Radiotherapy plus concomitant and adjuvant temozolomide for glioblastoma. N Engl J Med 352, 987-996.
Suva et al. (2014). Reconstructing and reprogramming the tumor-propagating potential of glioblastoma stem-like cells. Cell 157, 580-594.
TCGA (2008). Comprehensive genomic characterization defines human glioblastoma genes and core pathways. Nature 455, 1061-1068.
Trousseau, A. (1865). Phlegmasia Alba dolens. Clinique medicule de l'Hotel-Dieu de Paris 3, 94.
Unruh et al. (2019). Methylation-dependent Tissue Factor Suppression Contributes to the Reduced Malignancy of IDH1-mutant Gliomas. Clin Cancer Res 25, 747-759.
van den Berg, Y. W., Osanto, S., Reitsma, P. H., and Versteeg, H. H. (2012). The relationship between tissue factor and cancer progression: insights from bench and bedside. Blood 119, 924-932.
Varn et al. (2022). Glioma progression is shaped by genetic evolution and microenvironment interactions. Cell 185, 2184-2199 e2116.
Wang, B., Kohli, J., and Demaria, M. (2020). Senescent Cells in Cancer Therapy: Friends or Foes? Trends Cancer.
Wang et al. (2019). Inducing and exploiting vulnerabilities for the treatment of liver cancer. Nature 574, 268-272.
Wang et al. (2017). Tumor Evolution of Glioma-Intrinsic Gene Expression Subtypes Associates with Immunological Changes in the Microenvironment. Cancer Cell 32, 42-56 e46.
Wiley et al. (2019). SILAC Analysis Reveals Increased Secretion of Hemostasis-Related Factors by Senescent Cells. Cell Rep 28, 3329-3337 e3325.
Winchell, H. S., Anderson, A. C., and Pollycove, M. (1964). Radiation-Induced Hemorrhagic Diathesis in Dogs Unassociated with Thrombocytopenia: Association with an Intravascular Protein-Polysaccharide Particle. Blood 23, 186-192.
Xie et al. (2022). Quiescent human glioblastoma cancer stem cells drive tumor initiation, expansion, and recurrence following chemotherapy. Dev Cell 57, 32-46 e38.
Yoo et al. (2018). Proinvasive extracellular matrix remodeling in tumor microenvironment in response to radiation. Oncogene 37, 3317-3328.
Zhang et al. (2011). FoxM1 promotes beta-catenin nuclear localization and controls Wnt target-gene expression and glioma tumorigenesis. Cancer Cell 20, 427-442.
1. Lee, et al. (2006). Tumor stem cells derived from glioblastomas cultured in bFGF and EGF more closely mirror the phenotype and genotype of primary tumors than do serum-cultured cell lines. Cancer Cell 9, 391-403.
2. Kim et al. (2013). Phosphorylation of EZH2 activates STAT3 signaling via STAT3 methylation and promotes tumorigenicity of glioblastoma stem-like cells. Cancer Cell 23, 839-852.
3. Jacob et al. (2020). A Patient-Derived Glioblastoma Organoid Model and Biobank Recapitulates Inter- and Intra-tumoral Heterogeneity. Cell 180, 188-204 e122.
4. Hubert et al., (2016). A Three-Dimensional Organoid Culture System Derived from Human Glioblastomas Recapitulates the Hypoxic Gradients and Cancer Stem Cell Heterogeneity of Tumors Found In Vivo. Cancer Res 76, 2465-2477.
5. Pine et al. (2020). Tumor Microenvironment Is Critical for the Maintenance of Cellular States Found in Primary Glioblastomas. Cancer Discov 10, 964-979.
6. Hambardzumyan et al., (2009). Modeling Adult Gliomas Using RCAS/t-va Technology. Transl Oncol 2, 89-95.
7. Mathieu et al., (2008). Combining bevacizumab with temozolomide increases the antitumor efficacy of temozolomide in a human glioblastoma orthotopic xenograft model. Neoplasia 10, 1383-1392.
8. Brake et al., (2019). Assessing Blood Clotting and Coagulation Factors in Mice. Curr Protoc Mouse Biol 9, e61.
9. Debacq-Chainiaux et al., (2009). Protocols to detect senescence-associated beta-galactosidase (SA-betagal) activity, a biomarker of senescent cells in culture and in vivo. Nat Protoc 4, 1798-1806.
10. Neftel et al. (2019). An Integrative Model of Cellular States, Plasticity, and Genetics for Glioblastoma. Cell 178, 835-849 e821.
11. Verhaak et al. (2010). Integrated genomic analysis identifies clinically relevant subtypes of glioblastoma characterized by abnormalities in PDGFRA, IDH1, EGFR, and NF1. Cancer Cell 17, 98-110.
12. Ben-Porath et al., (2008). An embryonic stem cell-like gene expression signature in poorly differentiated aggressive human tumors. Nat Genet 40, 499-507.
13. Xie et al., (2022). Quiescent human glioblastoma cancer stem cells drive tumor initiation, expansion, and recurrence following chemotherapy. Dev Cell 57, 32-46 e38.
14. Bhat et al. (2013). Mesenchymal differentiation mediated by NF-kappaB promotes radiation resistance in glioblastoma. Cancer Cell 24, 331-346.
15. Tasdemir et al. (2016). BRD4 Connects Enhancer Remodeling to Senescence Immune Surveillance. Cancer Discov 6, 612-629.
16. Coppe et al., (2008). Senescence-associated secretory phenotypes reveal cell-nonautonomous functions of oncogenic RAS and the p53 tumor suppressor. PLoS Biol 6, 2853-2868.
17. Fridman, A. L., and Tainsky, M. A. (2008). Critical pathways in cellular senescence and immortalization revealed by gene expression profiling. Oncogene 27, 5975-5987.
18. Magnus, N., Gerges, N., Jabado, N., and Rak, J. (2013). Coagulation-related gene expression profile in glioblastoma is defined by molecular disease subtype. J Thromb Haemost 11, 1197-1200.
19. Brennan et al. (2013). The somatic genomic landscape of glioblastoma. Cell 155, 462-477.
20. Eckel-Passow et al. (2015). Glioma Groups Based on 1p/19q, IDH, and TERT Promoter Mutations in Tumors. N Engl J Med 372, 2499-2508.
21. Varn et al. (2022). Glioma progression is shaped by genetic evolution and microenvironment interactions. Cell 185, 2184-2199 e2116.
22. Wang et al. (2017). Tumor Evolution of Glioma-Intrinsic Gene Expression Subtypes Associates with Immunological Changes in the Microenvironment. Cancer Cell 32, 42-56 e46.
23. Mercurio et al., (1997). IKK-1 and IKK-2: cytokine-activated IkappaB kinases essential for NF-kappaB activation. Science 278, 860-866. 10.1126/science.278.5339.860.
24. Toso et al., (2002). A frequent human coagulation Factor VII mutation (A294V, c152) in loop 140 s affects the interaction with activators, tissue factor and substrates. Biochem J 363, 411-416. 10.1042/0264-6021:3630411.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, medicine, and molecular biology or related fields are intended to be within the scope of the following claims.

We claim:

1. A composition comprising: a human delta Factor VII construct, or nucleic acid sequence encoding said human delta Factor VII construct,
wherein said human delta Factor VII construct comprises:
a) a human Factor VII Propeptide domain, or portion thereof with substantially the same activity,
b) a human Factor VII EGF-1 domain, or portion thereof with substantially the same activity,
and wherein said human delta Factor VII construct lacks a Peptidase S1 domain or lacks a functional Peptidase S1 domain, and
wherein optionally said human delta Factor VII construct is less than 225 amino acids in length.

2. The composition of claim 1, wherein said human delta Factor VII construct: i) binds human coagulation factor III (F3), ii) lacks pro-coagulation activity in an in vitro coagulation assay, and/or iii) induces ubiquitin-mediated degradation of human F3 proteins in cell culture.

3. The composition of claims 1 or 2, wherein said human Factor VII Propeptide domain comprises the amino acid sequence of SEQ ID NO:6, or at least 30 consecutive amino acids from SEQ ID NO:6, or SEQ ID NO:6 with one, two, three, four, or five amino acid deletions or conservative amino acid changes.

4. The composition of any of claims 1-3, wherein said human Factor VII EGF-1 domain comprises the amino acid sequence of SEQ ID NO:8, or at least 30 consecutive amino acids from SEQ ID NO:8, or SEQ ID NO:8 with one, two, three, four, or five amino acid deletions or conservative amino acid changes.

5. The composition of any of claims 1-4, wherein said human delta Factor VII construct further comprises: c) a human Factor VII GLA domain, or portion thereof with substantially the same activity.

6. The composition of claim 5, wherein said human Factor VII GLA domain comprises the amino acid sequence of SEQ ID NO:7, or at least 30 consecutive amino acids from SEQ ID NO:7, or SEQ ID NO:7 with one, two, three, four, or five amino acid deletions or conservative amino acid changes.

7. The composition of any of claims 1-6, wherein said human delta Factor VII construct further comprises: c) a human Factor VII EGF-2 domain, or portion thereof with substantially the same activity.

8. The composition of claim 7, wherein said human Factor VII EGF-2 domain comprises the amino acid sequence of SEQ ID NO:9, or at least 30 consecutive amino acids from SEQ ID NO:9, or SEQ ID NO:9 with one, two, three, four, or five amino acid deletions or conservative amino acid changes.

9. The composition of any of claims 1-8, wherein said human delta Factor VII construct further comprises: c) a human Factor VII GLA domain, or portion thereof with substantially the same activity, and d) a human Factor VII EGF-2 domain, or portion thereof with substantially the same activity.

10. The composition of any of claims 1-9, wherein said composition comprises said nucleic acid sequence, which is optionally present in an expression vector.

11. The composition of claim 10, wherein said composition further comprises a neuro progenitor cell, and wherein said nucleic acid sequence is present in said neuro progenitor cells.

12. The composition of any of claims 1-11, further comprising at least one of the following: Temozolomide (TMZ), an EGFR inhibitor, a MET inhibitor, and an AKT inhibitor.

13. The composition of any of claims 1-12, further comprising at least one of the following: a physiologically tolerable buffer, normal saline, lactated ringers, and dextrose solution.

14. The composition of any of claims 1-13, wherein said composition comprises said human delta Factor VII construct.

15. The composition of any of claims 1-14, wherein said human delta Factor VII construct comprises SEQ ID NO: 3.

16. The composition of claim 1, wherein said human delta Factor VII construct comprises SEQ ID NO: 4 or 5.

17. A method of treating cancer comprising:
treating a human subject with said delta Factor VII construct, or cell or expression vector containing said nucleic acid sequence encoding said delta Factor VII construct, as recited in any of claims 1-16,
wherein said human subject has cancer.

18. The method of claim 17, wherein said human subject has brain cancer.

19. The method of claim 17, wherein said human subject has glioblastoma (GBM).

20. The method of claim 17, wherein said subject has been subject to radiation therapy for cancer prior to said treating, and/or wherein said subject is further treated with Temozolomide (TMZ).

21. The method of claim 20, wherein said human subject has GBM cells, and wherein said treating reduces the levels of F3 in said GBM cells.

22. The method of claim 17, wherein said treating comprises intravenous administration.

23. The method of claim 22, wherein said intravenous administration is in the neck of said human subject.

24. The method of claim 17, wherein said treating reduces the level of F3 proteins in cancer cells of said subject.

25. The method of claim 17, wherein said treating induces ubiquitin-mediated degradation of human F3 proteins in cancer cells of said subject.

26. A kit or system comprising:
a) said composition of any of claims 1-16, and
b) an intracranial or intravenous therapeutic delivery device, syringe vial, or shipment container.

27. The kit or system of claim 26, wherein said composition is present in said intracranial therapeutic delivery device, or said syringe vial, or said shipment container.


SEQUENCE LISTING (9 sequences)

SEQ ID NO: 1 - protein, 466 aa, Homo sapiens
MVSQALRLLCLLLGLQGCLAAGGVAKASGGETRDMPWKPGPHRVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 2 - protein, 181 aa, Synthetic construct
AGGVAKASGGETRDMPWKPGPHRVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTENLYFQGHHHHHH

SEQ ID NO: 3 - protein, 168 aa, Synthetic construct
AGGVAKASGGETRDMPWKPGPHRVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCT

SEQ ID NO: 4 - protein, 123 aa, Synthetic construct
AGGVAKASGGETRDMPWKPGPHRVFVTQEEAHGVLHRRRRDGDQCASSPCQNGGSCKDQLQSYICFCLPAFEGRNCETHKDDQLICVNENGGCEQYCSDHTGTKRSCRCHEGYSLLADGVSCT

SEQ ID NO: 5 - protein, 122 aa, Synthetic construct
AGGVAKASGGETRDMPWKPGPHRVFVTQEEAHGVLHRRRRANAFLEELRPGSLERECKEEQCSFEEAREIFKDAERTKLFWISYSDGDQCASSPCQNGGSCKDQLQSYICFCLPAFEGRNCE

SEQ ID NO: 6 - protein, 40 aa, Homo sapiens
AGGVAKASGGETRDMPWKPGPHRVFVTQEEAHGVLHRRRR

SEQ ID NO: 7 - protein, 45 aa, Homo sapiens
ANAFLEELRPGSLERECKEEQCSFEEAREIFKDAERTKLFWISYS

SEQ ID NO: 8 - protein, 37 aa, Homo sapiens
DGDQCASSPCQNGGSCKDQLQSYICFCLPAFEGRNCE

SEQ ID NO: 9 - protein, 42 aa, Homo sapiens
DQLICVNENGGCEQYCSDHTGTKRSCRCHEGYSLLADGVSCT